(19) United States
(12) Patent Application Publication
(10) Pub. No.: US 2010/0278751 A1
(43) Pub. Date: Nov. 4, 2010

(54) RADIOLABELED 2-AMINO-4-ALKYL-6-(HALOALKYL)PYRIDINE COMPOUNDS AND THEIR USE IN DIAGNOSTIC IMAGING

(75) Inventors: Robert H. Mach, Eureka, MO (US); Dong Zhou, Ballwin, MO (US); Michael Welch, St. Louis, MO (US)

Correspondence Address: Zackson Law LLC, 1015 Locust Street Suite 750, St. Louis, MO, 63101-1324, US

(73) Assignee: WASHINGTON UNIVERSITY, St. Louis, MO (US)

(21) Appl. No.: 12/771,040

(22) Filed: Apr. 30, 2010

Related U.S. Application Data
(60) Provisional application No. 61/174,483, filed on Apr. 30, 2009.

(51) Int. Cl. A61K 49/04; C07D 401/04; A61P 43/00

(52) U.S. Cl. 424/9.44; 546/276.4

(57) ABSTRACT

Radiolabeled 2-amino-4-alkyl-6-(haloalkyl)pyridine compounds are disclosed. In some aspects, the compounds bind to inducible nitric oxide synthase (iNOS) with high specificity. In some configurations, a compound comprising a radioisotope can be used for diagnostic imaging of iNOS distribution in a mammalian subject such as a human, using a scanning method such as positron emission tomography (PET scanning). Methods of synthesis of the compounds are also disclosed.

### PRIORITY

This application claims the benefit of and the priority to U.S. Provisional Application No. 61/174,483, filed on Apr. 30, 2009, which is incorporated herein by reference in its entirety.

### STATEMENT REGARDING FEDERALLY SPONSORED RESEARCH OR DEVELOPMENT

This invention was made with Government support grant P01 HL13851 awarded by the National Institutes of Health. The government has certain rights in the invention.

### INTRODUCTION

Nitric oxide (NO) has been identified as an important mediator of diverse physiological processes (Moncada, S, et al. Pharmacol. Rev. 43: 109-142, 1991). Endogenous NO is generated by nitric oxide synthase (NOS) enzymes (Zhang, J., et al. J. Med. Chem. 39: 5110-5118, 1996). The NOS family of enzymes includes inducible nitric oxide synthase (iNOS), endothelial nitric oxide synthase (eNOS) and neuronal nitric oxide synthase (nNOS). iNOS is a key mediator of reactive oxygen species (ROS) formed under conditions of oxidative stress. However, overproduction of NO by iNOS is associated with several pathological diseases, including ischemia/reperfusion injury, septic shock, vascular dysfunction in diabetes, and transplant rejection.

Design and development of inhibitors selective for iNOS is currently of considerable interest in pharmaceutical research. Researchers have identified 2-aminopyridine derivatives as potential iNOS inhibitors (Hagmann, W. K., et al. Bioorg. Med. Chem. Lett. 10: 1975-1978, 2000; U.S. Pat. No. 5,972,975). However, health care professionals are in need not only of compounds that exhibit greater iNOS inhibition, but also a satisfactory method of imaging iNOS activity in vivo. One attempt includes use of an ¹²⁵I-labeled diphenyleneiodonium bisulfate as a probe for imaging by positron emission tomography (“PET” scanning), but the specificity of ¹²⁵I-labeled inhibitor was found not to be satisfactory (McCarthy, T. J., et al. J. Nucl. Med. 34: 89P, 1993). Also, a N-nitro-L-arginine [¹¹C]methyl ester, a NOS inhibitor, was developed, but due to its instability in vivo, it cannot be used as a PET radio tracer (Roeda, D., et al. Nucl. Med. Biol. 23: 509-512, 1996). We previously reported the evaluation of ¹¹C and ¹⁸F radiolabeled iNOS inhibitors, S—[¹¹C]methylisothiourea ([¹¹C]MITU) and S-(2-[¹⁸F]fluoroethyl)isothiourea ([¹⁸F]FEITU) as probes for imaging iNOS distribution by PET scanning, using as subjects LPS-pretreated rats and EAE-affected mice (Zhang, J., et al. J. Med. Chem. 39: 5110-5118, 1996; Zhang, J. et al. 1: 263-267, Nitric Oxide: Bio. and Chem. 1997). However, MITU and FEITU inhibit iNOS with IC₅₀ values of at least 140 nM. Accordingly, alternative radiolabeled iNOS inhibitors for use as radiotracers in PET imaging are needed.

### SUMMARY

The present inventors have developed a series of compounds which can be used as radiolabels for diagnostic imaging, in particular positron emission tomography (PET) imaging of iNOS distribution in a mammal. In some embodiments, a compound can inhibit iNOS enzymatic activity. In some embodiments, a compound can comprise a radioisotope, such as a positron emitter. Accordingly, a compound of the present teachings can include a radioisotope such as a ¹¹C, an ¹⁸F, a ⁷⁶Br or a ¹²³I.

In some embodiments, the present teachings disclose a radiolabeled 2-amino-4-alkyl-6-(haloalkyl)pyridine compound or salt thereof having a structure

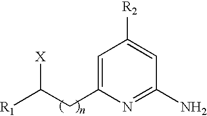

wherein R₁ can be H or C₁₋₅ alkyl, X can be a halogen, n can be an integer from 0 to 5, and R₂ can be C₁₋₅ alkyl, which inhibits iNOS with an IC₅₀ of less than 140 nM when measured against S-ethylisothiourea (SEITU), L-N(6)-(1-iminoethyl)lysine (L-NIL) and 2-aminopurine (2-AP) as standards.

In additional embodiments of the present teachings, the inventors disclose methods of imaging iNOS distribution in a mammal such as a human. These methods comprise administering to the mammal a radiolabeled 2-amino-4-alkyl-6-(haloalkyl)pyridine compound or salt thereof of the structure

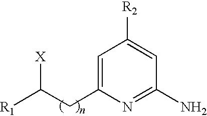

wherein R₁ can be H or C₁₋₅ alkyl, X can be a halogen, n can be an integer from 0 to 5, and R₂ can be C₁₋₅ alkyl, wherein the compound inhibits iNOS with an IC₅₀ of less than 140 nM when measured against SEITU, L-NIL and 2-AP as standards; and subjecting the mammal to PET scanning.

In some embodiments, a compound of the present teachings can be a radiolabeled 2-amino-4-alkyl-6-(haloalkyl)pyridine compound or salt thereof as disclosed herein, wherein R₁ can be H or C₁₋₅ alkyl, X can be a halogen, n can be an integer from 0 to 5, and R₂ can be C₁₋₅ alkyl, which inhibits iNOS with an IC₅₀ of less than or equal to about 57.6 nM when measured against S-ethylisothiourea (SEITU), L-N(6)-(1-iminoethyl)lysine (L-NIL) and 2-aminopurine (2-AP) as standards.

In some embodiments, a compound of the present teachings can be a radiolabeled 2-amino-4-alkyl-6-(haloalkyl)pyridine compound or salt thereof as disclosed herein, wherein R₁ can be H or C₁₋₅ alkyl, X can be a halogen, n can be an integer from 0 to 5, and R₂ can be C₁₋₅ alkyl, which inhibits iNOS with an IC₅₀ of less than or equal to about 54.3 nM when measured against S-ethylisothiourea (SEITU), L-N(6)-(1-iminoethyl)lysine (L-NIL) and 2-aminopurine (2-AP) as standards.

In some embodiments, a compound of the present teachings can be a radiolabeled 2-amino-4-alkyl-6-(haloalkyl)pyridine compound or salt thereof as disclosed herein, wherein R₁ can be H or CH₃, R₂ can be CH₃, X can be a halogen, and n can be an integer from 0 to 5, wherein the compound inhibits iNOS with an IC₅₀ of less than or equal to about 140 nM when measured against SEITU, L-NIL and 2-AP as standards.

In some embodiments, a compound of the present teachings can be a radiolabeled 2-amino-4-alkyl-6-(haloalkyl)pyridine compound or salt thereof as disclosed herein, wherein R₁ can be CH₃, n=1, R₂ can be CH₃, and X can be a halogen, wherein the compound inhibits iNOS with an IC₅₀ of less than 140 nM when measured against SEITU, L-NIL and 2-AP as standards.

In some embodiments, a compound of the present teachings can be a radiolabeled 2-amino-4-alkyl-6-(haloalkyl)pyridine compound or salt thereof as disclosed herein, wherein R₁ can be H, n=2, R₂ can be CH₃, and X can be a halogen, wherein the compound inhibits iNOS with an IC₅₀ of less than 140 nM when measured against SEITU, L-NIL and 2-AP as standards.

In some embodiments, a compound of the present teachings can be a radiolabeled 2-amino-4-alkyl-6-(haloalkyl)pyridine compound or salt thereof as disclosed herein, wherein R₁ can be ¹¹CH₃, X can be a halogen, n can be an integer from 0 to 5, and R₂ can be C₁₋₅ alkyl. In some configurations, such compounds or salts thereof can inhibit iNOS with an IC₅₀ of less than 140 nM when measured against SEITU, L-NIL and 2-AP as standards.

In some embodiments, a compound of the present teachings can be a radiolabeled 2-amino-4-alkyl-6-(haloalkyl)pyridine compound or salt thereof as disclosed herein, wherein R₂ can be ¹¹CH₃, R₁ can be H or C₁₋₅ alkyl, X can be a halogen, and n can be an integer from 0 to 5. In some configurations, such compounds or salts thereof can inhibit iNOS with an IC₅₀ of less than 140 nM when measured against SEITU, L-NIL and 2-AP as standards.

In some embodiments, a compound of the present teachings can be a radiolabeled 2-amino-4-alkyl-6-(haloalkyl)pyridine compound or salt thereof as disclosed herein, wherein X can be a halogen, wherein the halogen can be selected from an F, a Br and an I, R₁ is H or C₁₋₅ alkyl, n can be an integer from 0 to 5, and R₂ can be C₁₋₅ alkyl, wherein the compound inhibits iNOS with an IC₅₀ of less than 140 nM when measured against SEITU, L-NIL and 2-AP as standards.

In some embodiments, a compound of the present teachings can be a radiolabeled 2-amino-4-alkyl-6-(haloalkyl)pyridine compound or salt thereof as disclosed herein, wherein X can be a halogen, wherein the halogen can be selected from an F, a Br and an I, wherein the F can be an ¹⁸F, R₁ can be H or C₁₋₅ alkyl, n can be an integer from 0 to 5, and R₂ can be C₁₋₅ alkyl, wherein the compound inhibits iNOS with an IC₅₀ of less than 140 nM when measured against SEITU, L-NIL and 2-AP as standards.

In some embodiments, a compound of the present teachings can be a radiolabeled 2-amino-4-alkyl-6-(haloalkyl)pyridine compound or salt thereof as disclosed herein, wherein X can be a halogen, wherein the halogen can be selected from an F, a Br and an I, wherein the Br can be a ⁷⁶Br, R₁ can be H or C₁₋₅ alkyl, n can be an integer from 0 to 5, and R₂ can be C₁₋₅ alkyl, wherein the compound inhibits iNOS with an IC₅₀ of less than 140 nM when measured against SEITU, L-NIL and 2-AP as standards.

In some aspects, a compound of the present teachings can be a radiolabeled 2-amino-4-alkyl-6-(haloalkyl)pyridine compound or salt thereof as disclosed herein, wherein X can be a halogen, wherein the halogen can be selected from an F, a Br and an I, wherein the I can be a ¹²³I, R₁ can be H or C₁₋₅ alkyl, n can be an integer from 0 to 5, and R₂ can be C₁₋₅ alkyl. In some configurations, such compounds or salts thereof can inhibit iNOS with an IC₅₀ of less than 140 nM when measured against SEITU, L-NIL and 2-AP as standards.

In some aspects, a compound of the present teachings can be a radiolabeled 2-amino-4-alkyl-6-(haloalkyl)pyridine compound or salt thereof as disclosed herein, wherein n=1, R₁ can be CH₃, X can be ¹⁸F, and R₂ can be CH₃, wherein the compound inhibits iNOS with an IC₅₀ of less than 140 nM when measured against SEITU, L-NIL and 2-AP as standards.

In some embodiments, a compound of the present teachings can be a radiolabeled 2-amino-4-alkyl-6-(haloalkyl)pyridine compound or salt thereof as disclosed herein, wherein n=2, R₁ can H, X can be ¹⁸F, and R₂ can be CH₃, wherein the compound inhibits iNOS with an IC₅₀ of less than or equal to about 57.6 nM when measured against SEITU, L-NIL and 2-AP as standards.

In various aspects of the above embodiments, a radiolabeled 2-amino-4-alkyl-6-(haloalkyl)pyridine compound or salt thereof can include particular molecular species, such as

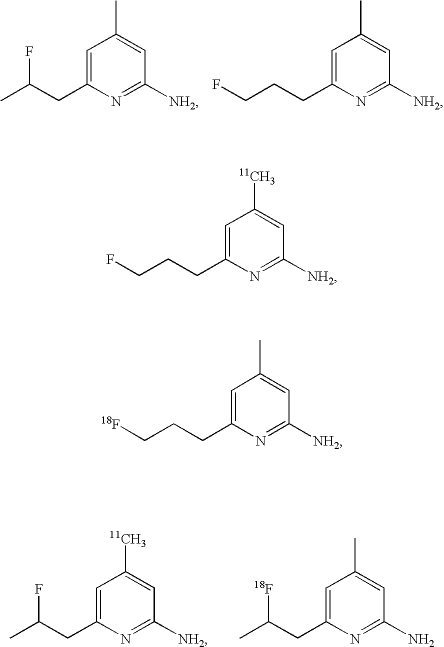

or a salt thereof.

In some embodiments of the present teachings, a salt can comprise a radiolabeled 2-amino-4-alkyl-6-haloalkylpyridine compound as disclosed herein which can serve as a cation (i.e., have positive charge, for example have an NH₃⁺ instead of NH₂) and an anion, such as a water soluble anion. In some configurations, the water soluble anion can be an anion of an organic acid, such as, without limitation, an oxalate.

In various aspects of the embodiments disclosing a salt comprising a radiolabeled 2-amino-4-alkyl-6-haloalkylpyridine compound and a water soluble anion, the compound can comprise, consist essentially of, or consist of particular molecular species, such as

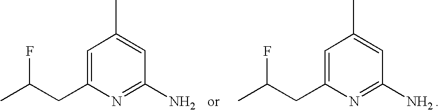

In various aspects of the embodiments, methods for the synthesis of the compounds disclosed herein are provided. In particular aspects, methods for the synthesis of iNOS inhibitors are provided. In further aspects, methods for the synthesis of iNOS inhibitor precursors are also provided.

The present disclosure comprises the following aspects:

1. A radiolabeled 2-amino-4-alkyl-6-(haloalkyl)pyridine compound or salt thereof of structure

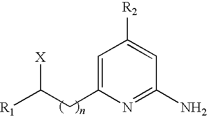

wherein R₁ is H or C₁₋₅ alkyl, X is a halogen, n is an integer from 0 to 5, and R₂ is C₁₋₅ alkyl, wherein the compound inhibits iNOS with an IC₅₀ of less than 140 nM when measured against SEITU, L-NIL and 2-AP as standards.
2. A radiolabeled 2-amino-4-alkyl-6-(haloalkyl)pyridine compound or salt thereof of structure

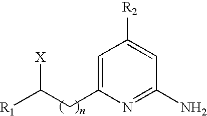

wherein R₁ is H or C₁₋₅ alkyl, X is a halogen, n is an integer from 0 to 5, and R₂ is C₁₋₅ alkyl, wherein the compound inhibits iNOS with an IC₅₀ less than that of SEITU, L-NIL or 2-AP and wherein at least one atom is a radioisotope.
3. A radiolabeled 2-amino-4-alkyl-6-haloalkylpyridine compound or salt thereof in accordance with claim 1, wherein the compound inhibits iNOS with an IC₅₀ of less than or equal to about 57.6 nM when measured against SEITU, L-NIL and 2-AP as standards.
4. A radiolabeled 2-amino-4-alkyl-6-haloalkylpyridine compound or salt thereof in accordance with aspect 1, wherein the compound inhibits iNOS with an IC₅₀ of less than or equal to about 54.3 nM when measured against SEITU, L-NIL and 2-AP as standards.
5. A radiolabeled 2-amino-4-alkyl-6-haloalkylpyridine compound or salt thereof in accordance with aspect 1, wherein R₁ is H or CH₃ and R₂ is CH₃.
6. A radiolabeled 2-amino-4-alkyl-6-haloalkylpyridine compound or salt thereof in accordance with aspect 4, wherein R₁ is CH₃ and n=1.
7. A radiolabeled 2-amino-4-alkyl-6-haloalkylpyridine compound or salt thereof in accordance with aspect 4, wherein R₁ is H and n=2.
8. A radiolabeled 2-amino-4-alkyl-6-haloalkylpyridine compound or salt thereof in accordance with aspect 1, wherein R₁ is ¹¹CH₃.
9. A radiolabeled 2-amino-4-alkyl-6-haloalkylpyridine compound or salt thereof in accordance with aspect 1, wherein R₂ is ¹¹CH₃.
10. A radiolabeled 2-amino-4-alkyl-6-haloalkylpyridine compound or salt thereof in accordance with aspect 1, wherein the halogen is selected from an F, a Br and an I.
11. A radiolabeled 2-amino-4-alkyl-6-haloalkylpyridine compound or salt thereof in accordance with aspect 10, wherein the F is an ¹⁸F.
12. A radiolabeled 2-amino-4-alkyl-6-haloalkylpyridine compound or salt thereof in accordance with aspect 10, wherein the Br is a ⁷⁶Br.
13. A radiolabeled 2-amino-4-alkyl-6-haloalkylpyridine compound or salt thereof in accordance with aspect 10, wherein the I is a ¹²³I.
14. A radiolabeled 2-amino-4-alkyl-6-haloalkylpyridine compound or salt thereof in accordance with aspect 5, wherein n=1, R₁ is a CH₃, X is an ¹⁸F, and R₂ is a CH₃.
15. A radiolabeled 2-amino-4-alkyl-6-haloalkylpyridine compound or salt thereof in accordance with aspect 5, wherein n=2, R₁ is an H, X is an ¹⁸F, and R₂ is CH₃.
16. A radiolabeled 2-amino-4-alkyl-6-haloalkylpyridine compound or salt thereof in accordance with aspect 1, wherein the compound is selected from the group consisting of

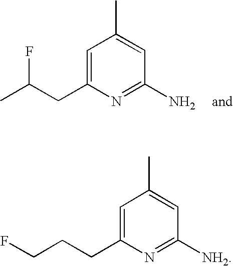

17. A radiolabeled 2-amino-4-alkyl-6-haloalkylpyridine compound or salt thereof in accordance with aspect 1, wherein the compound is selected from the group consisting of

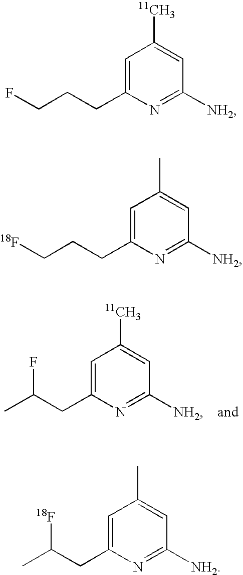

18. A salt comprising a radiolabeled 2-amino-4-alkyl-6-haloalkylpyridine compound in accordance with any one of aspects 1-17, and a water soluble anion.
19. A salt in accordance with aspect 18, wherein the water soluble anion is an anion of an organic acid.
20. A salt in accordance with aspect 18, wherein the anion is an oxalate.
21. A method of imaging iNOS distribution in a mammal, the method comprising:

administering to the mammal a radiolabeled 2-amino-4-alkyl-6-(haloalkyl)pyridine compound or salt thereof, or a salt, of any one of aspects 1-18; and

subjecting the mammal to PET scanning.

22. A method of imaging iNOS distribution in a mammal in accordance with aspect 21, wherein the radiolabeled 2-amino-4-alkyl-6-(haloalkyl)pyridine compound is selected from the group consisting of

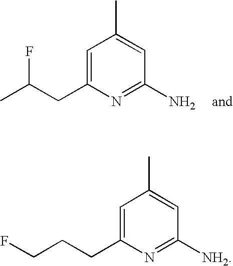

23. A method of imaging iNOS distribution in a mammal in accordance with aspect 21, wherein the radiolabeled 2-amino-4-alkyl-6-(haloalkyl)pyridine compound is selected from the group consisting of

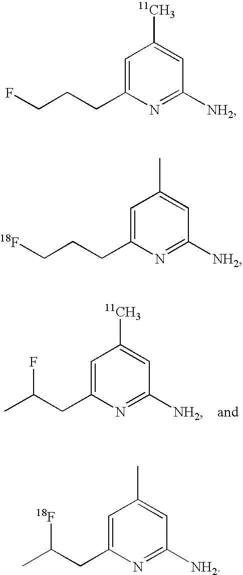

### BRIEF DESCRIPTION OF THE DRAWINGS

FIG. 1 illustrates reaction scheme I for synthesis of some compounds of the present teachings.

FIG. 2 illustrates reaction scheme II for synthesis of some compounds of the present teachings.

FIG. 3 illustrates structures and iNOS inhibition of some compounds of the present teachings.

FIG. 4 illustrates structures and iNOS inhibition of some compounds of the present teachings.

FIG. 5 illustrates structures and properties of some compounds of the present teachings.

FIG. 6 illustrates reaction scheme III for synthesis of some compounds of the present teachings.

FIG. 7 illustrates a PET/CT image of iNOS distribution in the pancreas of male ZDF rats. The PET image was acquired 1 hour after injection of [¹⁸F]compound 14.

FIG. 8 presents standard uptake values (SUV) of [¹⁸F]compound 14 radio tracer in the pancreas of the Zucker Diabetic Fatty (ZDF) rats and the pancreas of the lean rats.

FIG. 9 illustrates microPET images of iNOS distribution in the pancreas and liver of male Wistar rats injected with streptozotocin (STZ) and control male Wistar rats not injected with STZ. [¹⁸F]compound 14 was used as the radio tracer, and the microPET images of the rats were acquired 3 weeks and 8 weeks post injection.

FIG. 10 illustrates pancreas-to-liver ratios for iNOS distribution at 3 weeks and 8 weeks post STZ injection into male Wistar rats as well as for the control Wistar rats as determined by microPET imaging.

FIG. 11 presents a comparison of total lung radioactivity after injection of [¹⁸F]compound 14 in LPS-treated mice vs. control mice.

FIG. 12 depicts a representative Western blot of the levels of iNOS expression in lungs from the control and LPS-treated mice.

FIG. 13 shows a comparison of total lung radioactivity 1 hour after injection of [¹⁸F]compound 14 in control, LPS-treated, and LPS-treated-1400 W-blocked mice.

FIG. 14 shows whole-body microPET images of [¹⁸F]compound 14 in LPS treated and control mice, in transaxial and coronal views.

### DETAILED DESCRIPTION

The present inventors have developed a series of compounds which can be used as radiolabels for diagnostic imaging, in particular positron emission tomography (PET) imaging of iNOS distribution in a mammal. In some embodiments, a compound can inhibit iNOS enzymatic activity. In some embodiments, a compound can comprise a radioisotope, such as a positron emitter. Accordingly, a compound of the present teachings can include a radioisotope such as a ¹¹C, an ¹⁸F, a ⁷⁶Br or a ¹²³I. In some embodiments, a compound can comprise a radioisotope, and can be used as a radiotracer for imaging of iNOS distribution in a human or other mammal using positron emission tomography (PET). Furthermore, in some embodiments, a compound can comprise the radioisotope ¹²³I, and can be used for imaging of iNOS distribution using single photon emission computed tomography (SPECT) in a human or other mammal.

In some embodiments, a salt can comprise a compound of the present teachings which further carries charge and can be a cation, for example by comprising one or more additional protons, plus an anion such as, for example, a negatively charged organic anion such as, without limitation, oxalate. In some aspects, an anion can be a water-soluble anion such as, without limitation an organic anion such as, for example, oxalate.

Hence, in some aspects, the inventors provide methods of imaging iNOS distribution in a human or other animal. These methods comprise administering to the mammal a radiolabeled 2-amino-4-alkyl-6-haloaklylpyridine compound or salt thereof and subjecting the mammal to PET scanning.

Without limitation, imaging of iNOS distribution can be useful, for example, in the diagnosis of a disease characterized by abnormal accumulation of reactive oxygen species in an organ or tissue, such as, without limitation, diabetes. iNOS imaging can also be useful for monitoring progression of a therapeutic regime in a subject such as a human patient characterized by accumulation of reactive oxygen species in an organ or tissue. Thus, the present radiotracers and methods can be used, for example, by a medical professional to determine if a therapy is effective.

In some embodiments, a compound of the present teachings is a radiolabeled 2-amino-4-alkyl-6-(haloalkyl)pyridine compound or salt thereof having a structure

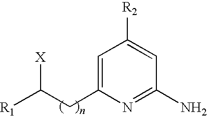

wherein R₁ is H or C₁₋₅ alkyl, X is a halogen, n is an integer from 0 to 5, and R₂ is C₁₋₅ alkyl, wherein the compound inhibits iNOS with an IC₅₀ of less than 140 nM when measured against S ethylisothiourea (SEITU), L-N(6)-(1-iminoethyl)lysine (L-NIL) and 2-aminopurine (2-AP) as standards.

In additional embodiments of the present teachings, the inventors disclose methods of imaging iNOS distribution in a mammal such as a human. These methods comprise administering to the mammal a radiolabeled 2-amino-4-alkyl-6-(haloalkyl)pyridine compound or salt thereof of the structure

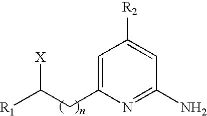

wherein R₁ is H or C₁₋₅ alkyl, X is a halogen, n is an integer from 0 to 5, and R₂ is a C₁₋₅ alkyl, wherein the compound inhibits iNOS with an IC₅₀ of less than 140 nM when measured against SEITU, L-NIL and 2-AP as standards; and subjecting the mammal to PET scanning.

In some embodiments, a compound of the present teachings can be a radiolabeled 2-amino-4-alkyl-6-(haloalkyl)pyridine compound or salt thereof as disclosed herein, wherein R₁ is H or C₁₋₅ alkyl, X is a halogen, n is an integer from 0 to 5, and R₂ is a C₁₋₅ alkyl, which inhibits iNOS with an IC₅₀ of less than or equal to about 57.6 nM when measured against S-ethylisothiourea (SEITU), L-N(6)-(1-iminoethyl)lysine (L-NIL) and 2-aminopurine (2-AP) as standards.

In some embodiments, a compound of the present teachings can be a radiolabeled 2-amino-4-alkyl-6-(haloalkyl)pyridine compound or salt thereof as disclosed herein, wherein R₁ is H or C₁₋₅ alkyl, X is a halogen, n is an integer from 0 to 5, and R₂ is a C₁₋₅ alkyl, which inhibits iNOS with an IC₅₀ of less than or equal to about 54.3 nM when measured against S-ethylisothiourea (SEITU), L-N(6)-(1-iminoethyl)lysine (L-NIL) and 2-aminopurine (2-AP) as standards.

In some embodiments, a compound of the present teachings is a radiolabeled 2-amino-4-alkyl-6-(haloalkyl)pyridine compound or salt thereof as disclosed herein, wherein R₁ is H or CH₃, R₂ is CH₃, X is a halogen, and n is an integer from 0 to 5, wherein the compound inhibits iNOS with an IC₅₀ of less than 140 nM when measured against SEITU, L-NIL and 2-AP as standards.

In some embodiments, a compound of the present teachings is a radiolabeled 2-amino-4-alkyl-6-(haloalkyl)pyridine compound or salt thereof as disclosed herein, wherein R₁ is CH₃, n=1, R₂ is CH₃, and X is a halogen, wherein the compound inhibits iNOS with an IC₅₀ of less than 140 nM when measured against SEITU, L-NIL and 2-AP as standards.

In some embodiments, a compound of the present teachings is a radiolabeled 2-amino-4-alkyl-6-(haloalkyl)pyridine compound or salt thereof as disclosed herein, wherein R₁ is H, n=2, R₂ is CH₃, and X is a halogen, wherein the compound inhibits iNOS with an IC₅₀ of less than or equal to about 140 nM when measured against SEITU, L-NIL and 2-AP as standards.

In some embodiments, a compound of the present teachings is a radiolabeled 2-amino-4-alkyl-6-(haloalkyl)pyridine compound or salt thereof as disclosed herein, wherein R₁ is ¹¹CH₃, X is a halogen, n is an integer from 0 to 5, and R₂ is C₁₋₅ alkyl, wherein the compound inhibits iNOS with an IC₅₀ of less than 140 nM when measured against SEITU, L-NIL and 2-AP as standards.

In some embodiments, a compound of the present teachings is a radiolabeled 2-amino-4-alkyl-6-(haloalkyl)pyridine compound or salt thereof as disclosed herein, wherein R₂ is ¹¹CH₃, R₁ is H or C₁₋₅ alkyl, X is a halogen, and n is an integer from 0 to 5, wherein the compound inhibits iNOS with an IC₅₀ of less than 140 nM when measured against SEITU, L-NIL and 2-AP as standards.

In some embodiments, a compound of the present teachings is a radiolabeled 2-amino-4-alkyl-6-(haloalkyl)pyridine compound or salt thereof as disclosed herein, wherein X is a halogen, wherein the halogen is selected from an F, a Br and an I, R₁ is H or C₁₋₅ alkyl, n is an integer from 0 to 5, and R₂ is C₁₋₅ alkyl, wherein the compound inhibits iNOS with an IC₅₀ of less than 140 nM when measured against SEITU, L-NIL and 2-AP as standards.

In some embodiments, a compound of the present teachings is a radiolabeled 2-amino-4-alkyl-6-(haloalkyl)pyridine compound or salt thereof as disclosed herein, wherein X is a halogen, wherein the halogen is selected from an F, a Br and an I, wherein the F is an ¹⁸F, R₁ is H or C₁₋₅ alkyl, n is an integer from 0 to 5, and R₂ is C₁₋₅ alkyl, wherein the compound inhibits iNOS with an IC₅₀ of less than 140 nM when measured against SEITU, L-NIL and 2-AP as standards.

In some embodiments, a compound of the present teachings is a radiolabeled 2-amino-4-alkyl-6-(haloalkyl)pyridine compound or salt thereof as disclosed herein, wherein X is a halogen, wherein the halogen is selected from an F, a Br and an I, wherein the Br is a ⁷⁶Br, R₁ is H or C₁₋₅ alkyl, n is an integer from 0 to 5, and R₂ is C₁₋₅ alkyl, wherein the compound inhibits iNOS with an IC₅₀ of less than 140 nM when measured against SEITU, L-NIL and 2-AP as standards.

In some embodiments, a compound of the present teachings is a radiolabeled 2-amino-4-alkyl-6-(haloalkyl)pyridine compound or salt thereof as disclosed herein, wherein X is a halogen, wherein the halogen is selected from an F, a Br and an I, wherein the I is a ¹²³I, R₁ is H or C₁₋₅ alkyl, n is an integer from 0 to 5, and R₂ is C₁₋₅ alkyl, wherein the compound inhibits iNOS with an IC₅₀ of less than 140 nM when measured against SEITU, L-NIL and 2-AP as standards.

In some embodiments, a compound of the present teachings is a radiolabeled 2-amino-4-alkyl-6-(haloalkyl)pyridine compound or salt thereof as disclosed herein, wherein n=1, R₁ is a CH₃, X is an ¹⁸F, and R₂ is a CH₃, wherein the compound inhibits iNOS with an IC₅₀ of less than 140 nM when measured against SEITU, L-NIL and 2-AP as standards.

In some embodiments, a compound of the present teachings is a radiolabeled 2-amino-4-alkyl-6-(haloalkyl)pyridine compound or salt thereof as disclosed herein, wherein n=2, R₁ is an H, X is an ¹⁸F, and R₂ is CH₃, wherein the compound inhibits iNOS with an IC₅₀ of less than 140 nM when measured against SEITU, L-NIL and 2-AP as standards.

In various aspects of the above embodiments, a radiolabeled 2-amino-4-alkyl-6-(haloalkyl)pyridine compound or salt thereof can include particular can include particular molecular species, such as

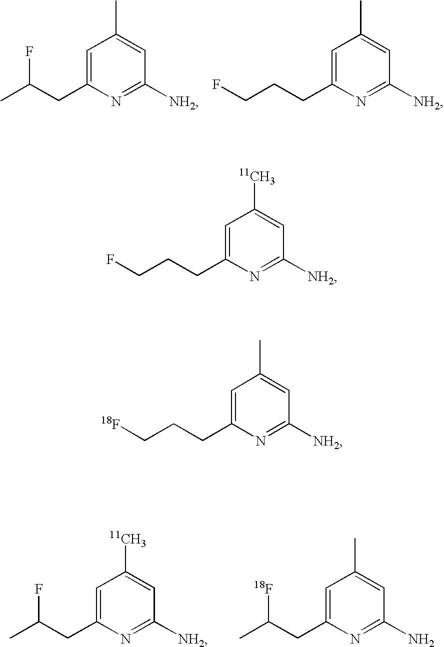

or a salt thereof.

In some embodiments of the present teachings, a salt can comprise a radiolabeled 2-amino-4-alkyl-6-haloalkylpyridine compound as disclosed herein and a water soluble anion. In some configurations, the water soluble anion is an anion of an organic acid, such as, without limitation, an oxalate.

In various aspects of the embodiments disclosing a salt comprising a radiolabeled 2-amino-4-alkyl-6-haloalkylpyridine compound and a water soluble anion, can include particular molecular species, such as

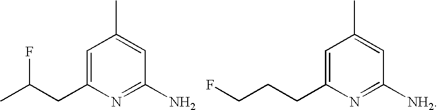

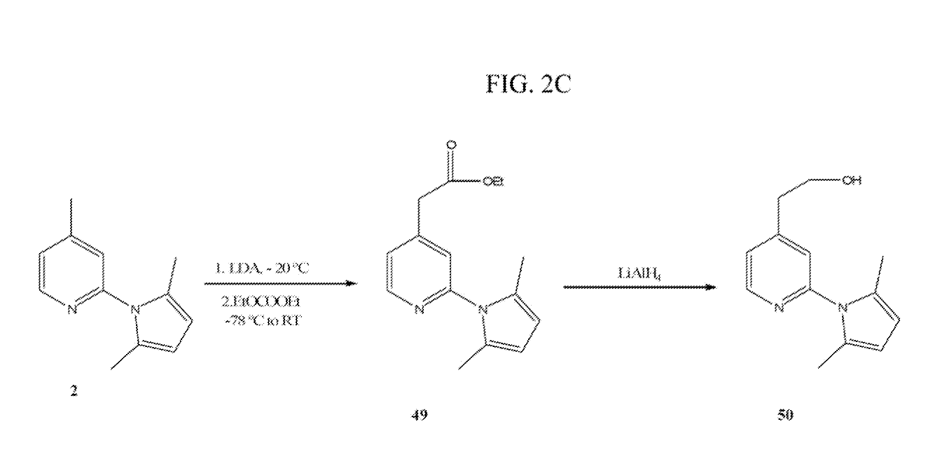

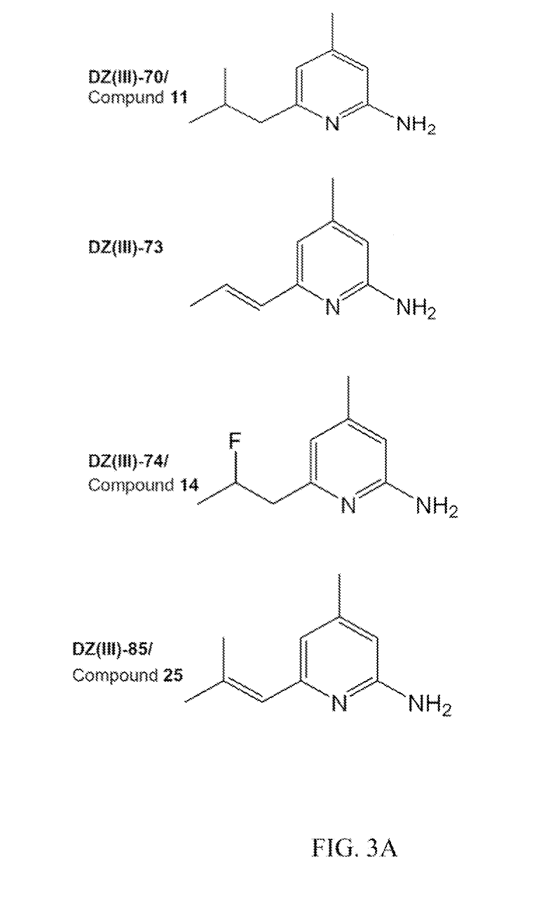

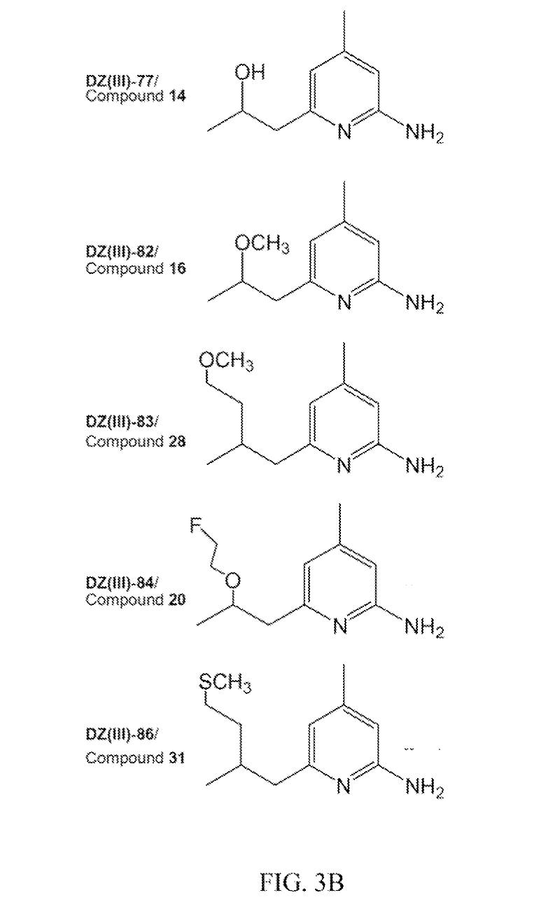

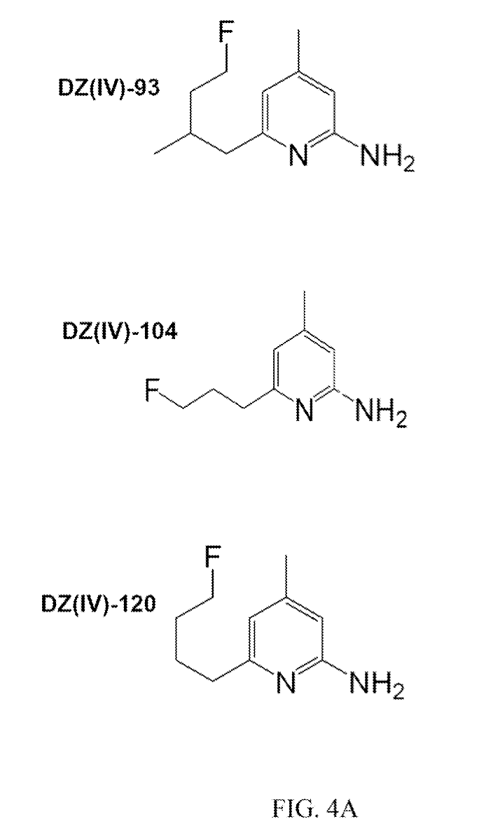

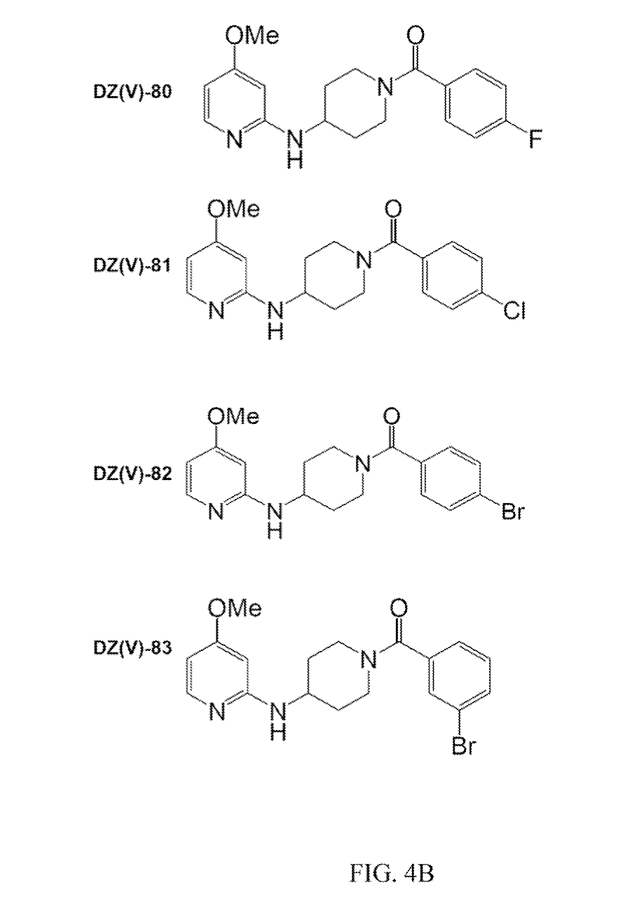

The present inventors have synthesized several 2-aminopyridine compounds which can inhibit iNOS as shown in FIG. 3 and FIG. 4. Two of those compounds, compound 14 and compound 52, exhibited the greatest inhibition of iNOS, as show in FIG. 5, and exhibit an IC₅₀ of less than 190 nM when measured against SEITU, L-NIL and 2-AP as standards.

### EXAMPLES

Our studies indicate that various compounds of the present teachings, including [¹⁸F][compound 14], are acceptable agents for detecting and imaging iNOS distribution with PET. The following examples are illustrative of the various embodiments of the present teachings. The examples are not intended to limit the scope of the claims. The methods described herein utilize laboratory techniques well known to skilled artisans, and guidance can be found in laboratory manuals and textbooks such as Sambrook, J., et al., Molecular Cloning: A Laboratory Manual, 3rd ed. Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998; and Harlow, E., Using Antibodies: A Laboratory Manual, Cold Spring Harbor Laboratory Press, Cold Spring Harbor, N.Y., 1999; Hedrickson et al., Organic Chemistry 3rd edition, McGraw Hill, New York, 1970; Carruthers, W., and Coldham, I., Modern Methods of Organic Synthesis (4th Edition), Cambridge University Press, Cambridge, U.K., 2004; Curati, W. L., Imaging in Oncology, Cambridge University Press, Cambridge, U.K., 1998; Welch, M. J., and Redvanly, C. S., eds. Handbook of Radiopharmaceuticals: Radiochemistry and Applications, J. Wiley, New York, 2003.

In the experiments described herein, all reagents were purchased from commercial suppliers and used without further purification unless otherwise stated. All reactions were carried out by standard air-free and moisture-free techniques under an inert argon atmosphere with dry solvents unless otherwise stated. Flash column chromatography was conducted using Scientific Adsorbents, Inc. silica gel, 60 A, “40 Micron Flash” (32-63 μm). Melting points were determined using MEL-TEMP 3.0 apparatus and uncorrected. ¹H NMR spectra were recorded on a Varian Unitey-300 (300 MHz) NMR spectrometer. All chemical shifts were reported a part per million (ppm) downfield from tetramethylsilane (TMS). Chloroform-d was used as solvent and the solvent peak at δ 7.25 ppm was used as an internal standard. All coupling constants (J) are given in Hertz (Hz). Splitting patterns are typically described as follows: s, singlet; d, doublet; t, triplet; m, multiplet.

### Example 1

This example illustrates synthesis of compound 2.

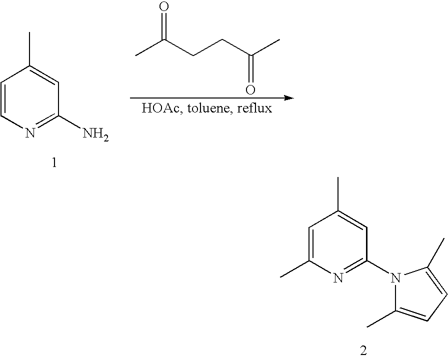

Compound 2 was synthesized as reported in Hagmann. et al. Bioorg. Med. Chem. Lett. 10, 1975-1976, 2000.

### Example 2

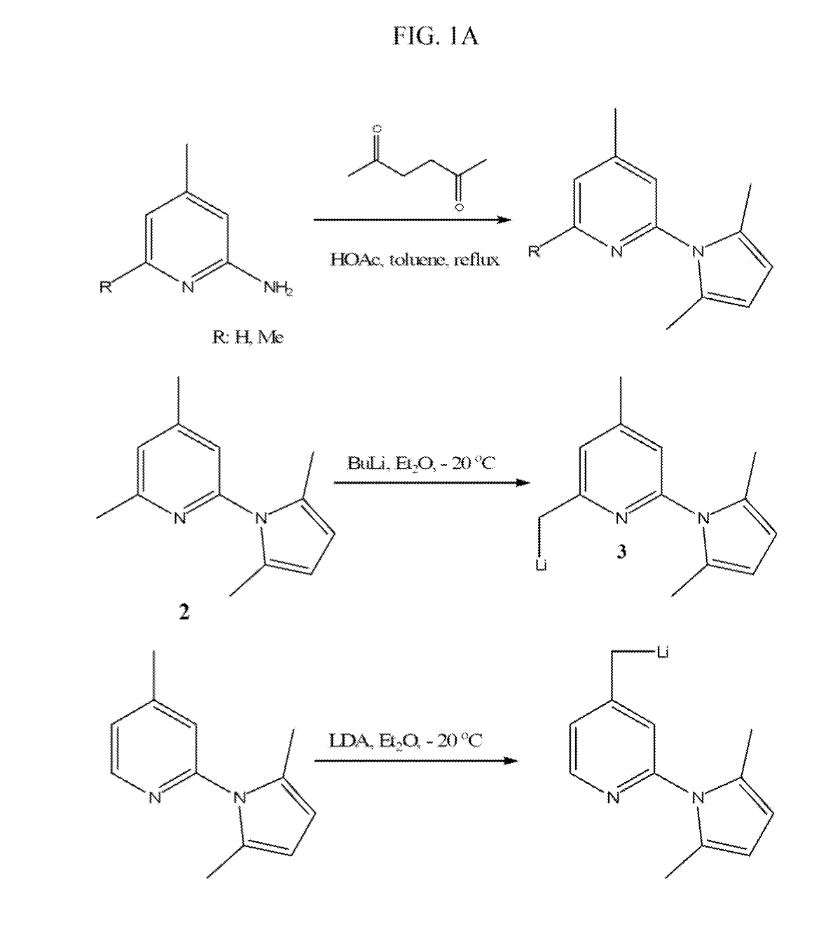

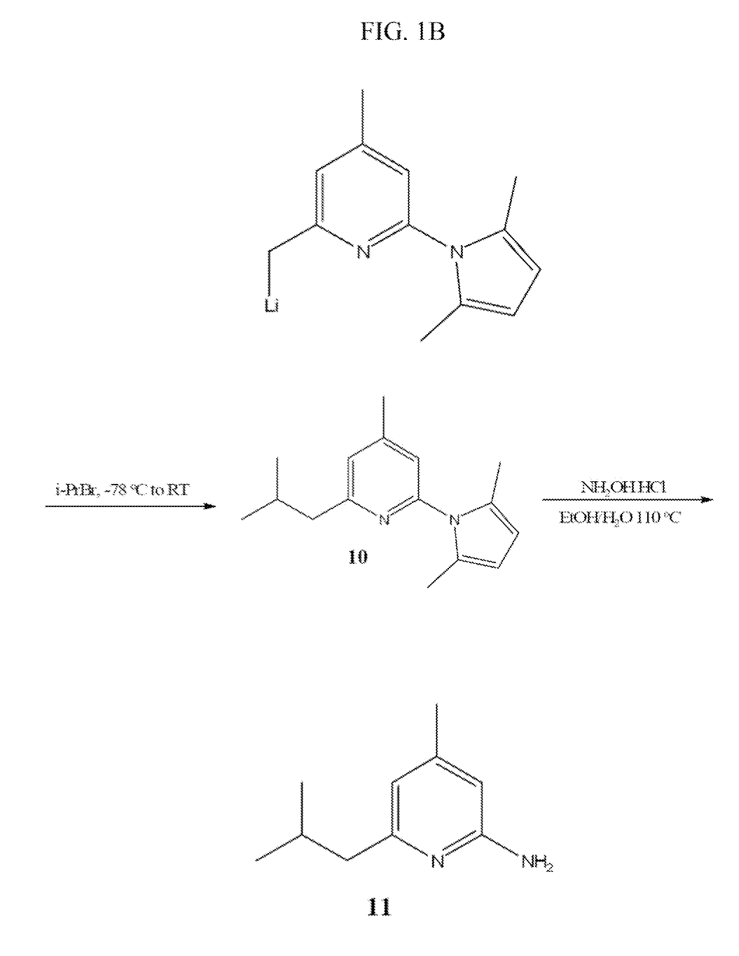

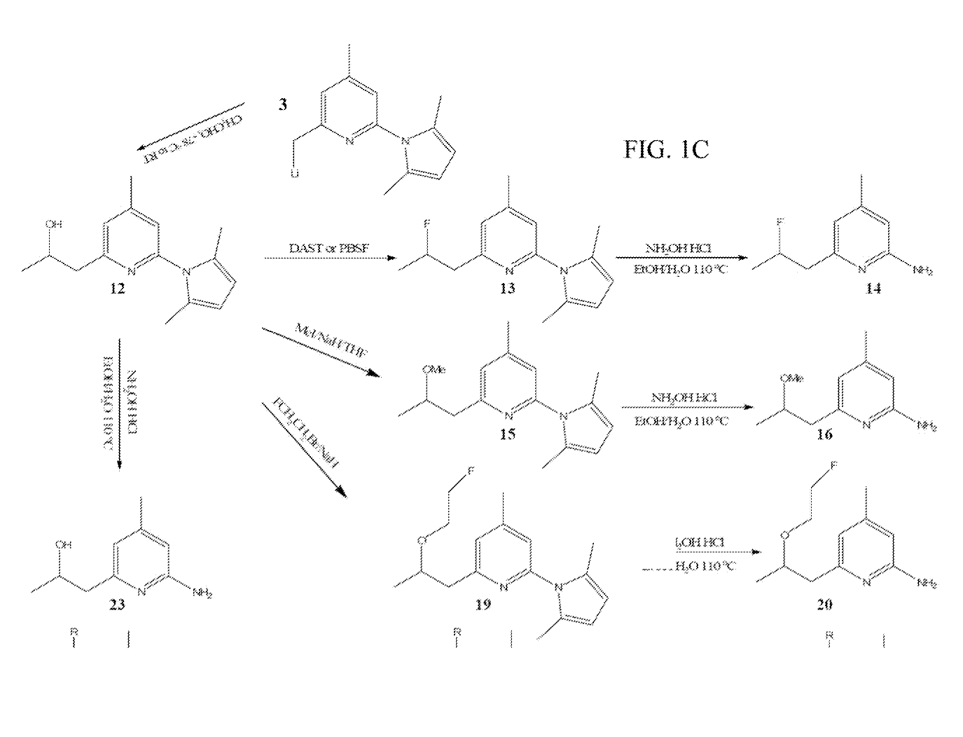

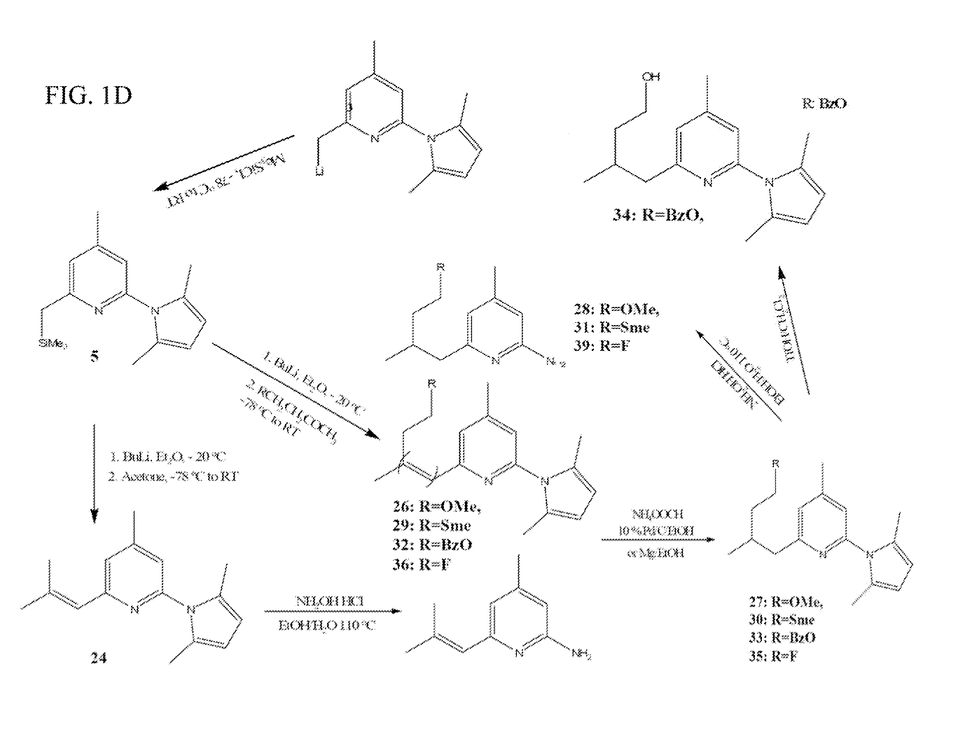

This example illustrates synthesis of compound 3, as shown in Reaction Scheme I (FIG. 1).

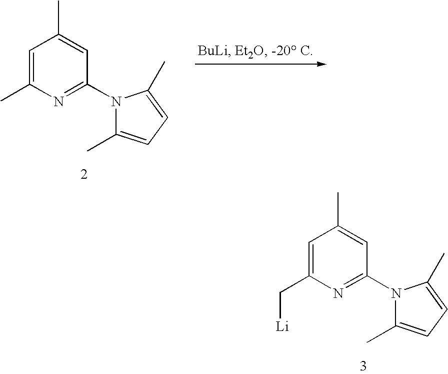

To synthesize compound 3, 6.9 ml (10 mmol) of n-butyllithium (n-BuLi) (1.6 M in Hexane) was added dropwise into a solution of 2.0 g (10 mmol) compound 2 in 20 ml dry diethyl ether (Et₂O) at −20° C. Orange crystals precipitated gradually, and the mixture was stirred for 1 hour to complete the reaction. Compound 3 is shown in both Reaction Scheme I and Reaction Scheme II as the compound from which the remaining synthesis reactions proceed.

### Example 3

This example illustrates synthesis of compound 10, as shown in Reaction Scheme I (FIG. 1).

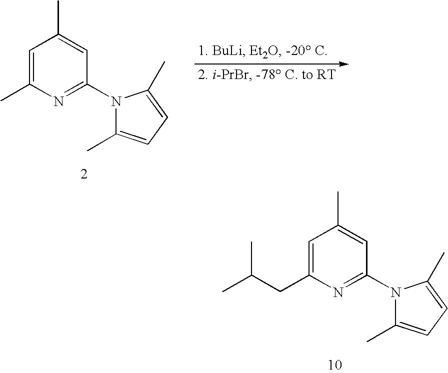

To synthesize compound 10, compound 3 was synthesized by adding 6.9 ml (10 mmol) of n-butyllithium (n-BuLi) (1.6 M in Hexane) dropwise into a solution of 2.0 g (10 mmol) compound 2 in 20 ml dry diethyl ether (Et₂O) at −20° C. Orange crystals precipitated gradually, and the mixture was stirred for 1 hour to complete the reaction. After the reaction mixture was cooled down to −78° C., 1.23 g (10 mmol) of isopropyl bromide (i-PrBr) in 10 ml of Et₂O was added via a syringe. Then, the reaction mixture was allowed to warm up to room temperature and continued to stir for one hour. After completion of the reaction according to thin layer chromatography (TLC), the mixture was treated with water, and the aqueous layer was extracted with ethyl acetate. The organic layers were combined and dried over sodium sulfate (Na₂SO₄). Solvents were removed under reduced pressure, the residue was purified with silica gel chromatograph using 1:15 EA/Hexane to afford 0.96 g (40%) of compound 10 as colorless liquid. The ¹H NMR spectrum (CD₃Cl, 300 MHz) of the purified product was: δ: 6.94 (s, 1H), 6.84 (s, 1H), 5.87 (s, 2H), 2.64 (d, 2H, J=7.2 Hz), 2.38 (s, 3H), 2.15 (m, 1H), 2.10 (s, 6H), 0.92 (d, 6H, J=6.9 Hz).

### Example 4

This example illustrates synthesis of compound 11, as shown in Reaction Scheme I (FIG. 1).

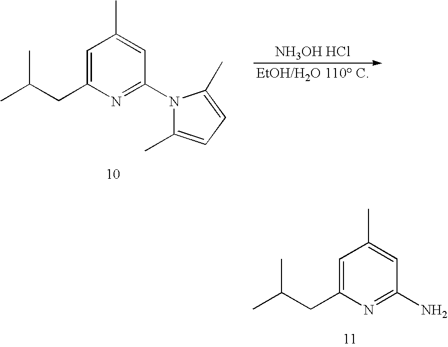

To synthesize compound 11, 0.96 g (4.0 mmol) of compound 10 and 10 ml of 4 M NH₂OH.HCl in 1:1 EtOH/H₂O were added into a 50 ml round-bottom flask equipped with a magnetic stirring bar and condenser. The reaction mixture was refluxed in a 110° C. oil bath and the reaction was checked by TLC. Upon the completion of the reaction in about 3 hours, the reaction mixture was treated with 10 ml saturated sodium carbonate (Na, CO₃) and extracted with 3×20 ml dichloromethane (CH₂Cl₂). The organic solution was washed with 2×20 ml brine, dried over Na₂SO₄. After removal of solvents under reduced pressure, the crude product was purified with silica gel chromatograph using 1:1 EA/Hexane to afford 0.35 g (53%) of compound 11 as slightly yellow solid with a melting point of 59-61° C. The ¹H NMR spectrum (CD₃Cl, 300 MHz) of the purified product was: δ: 6.32 (s, 1H), 6.17 (s, 1H), 4.43 (s, br, 2H), 2.43 (d, 2H, J=7.2 Hz), 2.20 (s, 3H), 2.04 (m, 1H), 0.92 (d, 6H, J=6.6 Hz). Oxalate of compound 11 (1:1) (melting point of 142.3-143.0° C.).

### Example 5

This example illustrates synthesis of compound 12, as shown in Reaction Scheme I (FIG. 1).

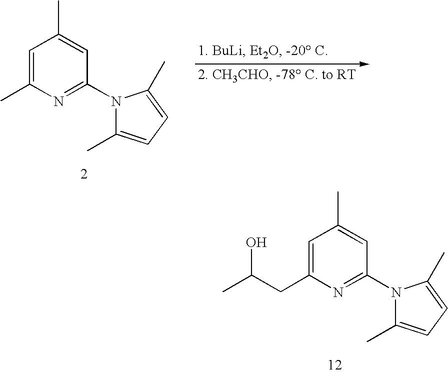

To synthesize compound 12, 2 ml (35.6 mmol) of acetaldehyde (CH₃CHO) was added via a syringe into an Et₂O solution of lithium of compound 2 at −78° C., which was made from 5.6 g (28.0 mmol) of compound 2, 19.5 ml (31.2 mmol) of n-BuLi (1.6 M in Hexane) in 30 ml Et₂O, was added. The reaction mixture was allowed to warm up to room temperature and continued to stir for 10 minutes. Then, the reaction mixture was treated with water (H₂O), and extracted with ethyl acetate. The organic solution was washed with brine, dried over Na₂SO₄ and the solvent was evaporated under reduced pressure. The crude product was purified with silica gel chromatograph using 1:4 EA/Hexane to afford 5.6 g (80%) of compound 12 as colorless liquid. The ¹H NMR spectrum (CD₃Cl, 300 MHz) of the purified product was: δ: 6.98 (s, 1H), 6.90 (s, 1H), 5.88 (s, 2H), 4.51 (s, br, 1H), 4.22 (m, 1H), 2.88 (m, 2H), 2.40 (s, 3H), 2.12 (s, 6H), 1.25 (d, 3H, J=6.3 Hz).

### Example 6

This example illustrates synthesis of compound 13, particularly synthesis of compound 13 using diethylaminosulfur trifluoride (DAST) as a fluorinating agent, as shown in Reaction Scheme I (FIG. 1).

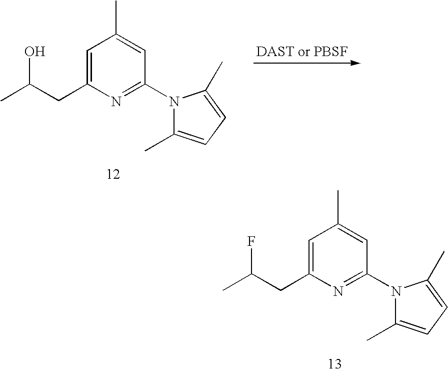

To synthesize compound 13, 0.4 ml (3.05 mmol) of DAST was added dropwise into a solution of 0.5 g (2.05 mmol) compound 12 in 5 ml CH₂Cl₂ at 0° C. The reaction mixture became brown in color, and compound 12 was gone within 30 minutes. 10 ml saturated NaHCO₃/H₂O solution was added to quench the reaction. The aqueous solution was extracted with 30 ml ethyl acetate, and all the organic solution was washed with 2×30 ml brine, dried over Na₂SO₄. Solvents were evaporated under reduced pressure and the crude product was purified with silica gel chromatograph using 1:10 EA/Hexane to afford 0.13 g (26%) of compound 13 as colorless liquid. The ¹H NMR spectrum (CD₃Cl, 300 MHz) of the purified product was: δ: 7.07 (s, 1H), 6.93 (s, 1H), 5.91 (s, 2H), 5.05-5.60 (m, 1H), 3.20 (m, 2H), 2.43 (s, 3H), 2.15 (s, 6H), 1.43 (dd, 3H, J=23.4, 6.3 Hz).

### Example 7

This example illustrates synthesis of compound 13, particularly synthesis of compound 13 using perfluorobutane sulfonyl fluoride (PBSF) as a fluorinating agent, as shown in Reaction Scheme I (FIG. 1).

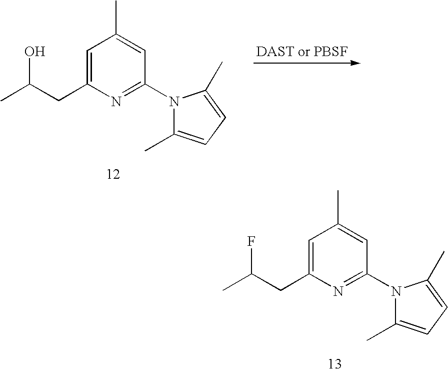

To synthesize compound 13, 0.74 ml PBSF, 1.73 ml NEt₃ and 0.67 ml (NEt₃)(HF)₃ were added into a solution of 0.5 g (2.05 mmol) of compound 12 in 5 ml CAN. The reaction mixture was stirred quickly and after 40 minutes, the reaction mixture became homogenous. Upon the completion of the reaction in 2.5 hours according to TLC, the solvent was evaporated under reduced pressure. Silica gel chromatograph purification of the residue using 1:10 EA/Hexane afforded 0.13 g (26%) of compound 13 as colorless liquid.

### Example 8

This example illustrates synthesis of compound 14, as shown in Reaction Scheme I (FIG. 1).

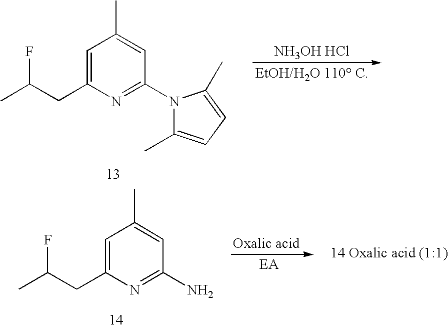

Compound 14 was synthesized from compound 13 using a standard deprotection procedure. Oxalate of compound 14 was prepared using 1:1 of compound 14 and oxalic acid in ethyl acetate as white solid. The ¹H NMR spectrum (CD₃Cl, 300 MHz) of the purified product was: δ: 6.42 (s, 1H), 6.21 (s, 1H), 4.90-5.20 (m, 1H), 4.39 (s, br, 2H), 2.70-3.00 (m, 2H), 2.21 (s, 3H), 1.39 (dd, 3H, J=24.0, 6.3 Hz).

### Example 9

This example illustrates synthesis of compound 15, as shown in Reaction Scheme I (FIG. 1).

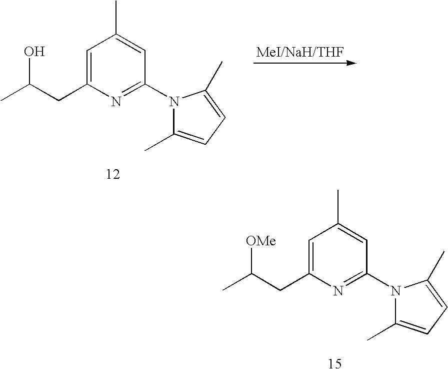

To synthesize compound 15, 0.3 g (12.6 mmol) 60% NaH in mineral oil was added into a solution of 0.9 g (3.68 mmol) of compound 12 in 20 ml freshly distilled THF. After 5 minutes, 0.5 ml (8.03 mmol) MeI was added, and the reaction mixture was stirred at room temperature. Upon the completion of the reaction in 5 hours, the reaction was quenched by 1 N HCl and neutralized with saturated Na₂CO₃. The product was extracted with 3×20 ml ethyl acetate and the organic solution was washed with brine, dried over MgSO₄. After the evaporation of solvents under reduced pressure, Silica gel chromatograph purification of the residue using 1:4 EA/Hexane afforded 0.89 g (94%) of compound 15 as liquid with slightly yellow color. The ¹H NMR spectrum (CD₃Cl, 300 MHz) of the purified product was: δ: 7.01 (s, 1H), 6.87 (s, 1H), 5.88 (s, 2H), 3.80-3.90 (m, 1H), 3.31 (s, 3H), 2.80-3.05 (m, 2H), 2.39 (s, 3H), 2.12 (s, 6H), 1.17 (d, 3H, J=6.0 Hz).

### Example 10

This example illustrates synthesis of compound 16, as shown in Reaction Scheme I (FIG. 1).

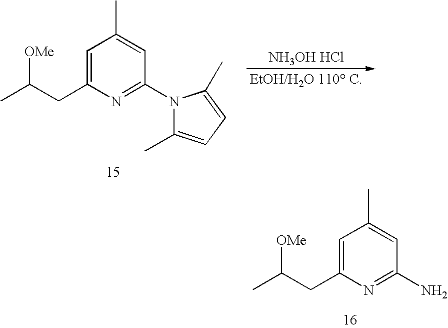

Compound 16 was synthesized from compound 15 using a standard deprotection procedure. Oxalate of compound 14 was prepared using 1:1 of compound 14 and oxalic acid in ethyl acetate as white solid. The ¹H NMR spectrum (CD₃Cl, 300 MHz) of the purified product was: δ: 6.37 (s, 1H), 6.15 (s, 1H), 4.44 (s, br, 2H), 3.72 (m, 1H), 3.30 (s, 3 h), 2.50-2.90 (m, 2H), 2.18 (s, 3H), 1.14 (d, 3H, J=6 Hz).

### Example 11

This example illustrates synthesis of compound 17.

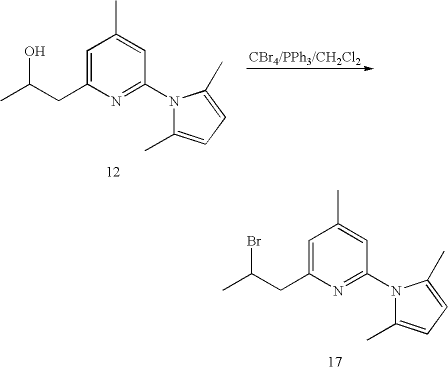

To synthesize compound 17, 2.18 g (8.3 mmol) PPh₃, followed by 2.77 g (8.3 mmol) CBr₄ was added into a solution of 1 g (4.09 mmol) of compound 12 in 20 ml CH₂Cl₂ at 0° C. The reaction was completed within 30 minutes. The reaction mixture was treated with 10 ml saturated NaHCO₃ and brine, and passed through a column of silica gel (25 cm×2 cm). The eluted solution was evaporated under reduced pressure, and the residue was purified by silica gel chromatograph using 1:15 EA/Hexane to afford 0.56 g (44%) of compound 17 as two isomers as slightly yellow color liquid. The ¹H NMR spectrum (CD₃Cl, 300 MHz) of the purified product was: δ: 7.02 (s, 1H), 7.01 (s, 1H), 6.92 (s, 2H), 5.89 (s, 4H), 4.6 (m, 2H), 3.2 (m, 4H), 2.42 (s, 6H), 2.11 (s, 12H), 1.78 (d, 3H, J=6.9 Hz), 1.58 (d, 3H, J=6.6 Hz).

### Example 12

This example illustrates synthesis of compound 19, as shown in Reaction Scheme I (FIG. 1).

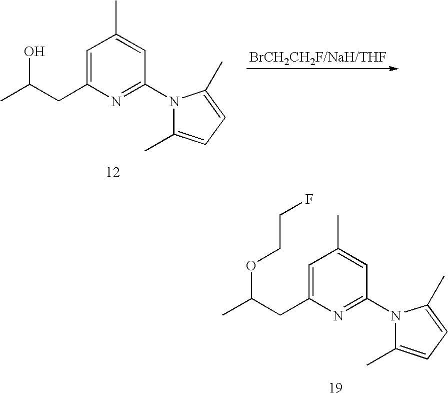

To synthesize compound 19, 0.35 g (8.8 mmol) 60% NaH in mineral oil and 1 ml (8 mmol) 1-bromo-2-fluoroethane were added into a solution of 1.0 g (4.1 mmol) compound 12 in 20 ml freshly distilled THF. The reaction mixture was stirred at room temperature for 6 hours, then at 60° C. overnight. NaH was filtered and the organic solution was washed with 1 N HCl, saturated Na₂CO₃, brine and dried over Na₂SO₄. Solvent was evaporated under reduced pressure and the residue was purified by silica gel chromatograph using 1:8 EA/Hexane to afford 0.37 g (31%) of compound 19 as colorless liquid. The ¹H NMR spectrum (CD₃Cl, 300 MHz) of the purified product was: δ: 7.05 (s, 1H), 6.87 (s, 1H), 5.88 (s, 2H), 4.36-4.55 (m, 2H), 3.98 (m, 1H), 3.53-3.67 (m, 2H), 2.82-3.09 (m, 2H), 2.39 (s, 3H), 2.10 (s, 6H), 1.20 (d, 3H, J=6.3 Hz).

### Example 13

This example illustrates synthesis of compound 20, as shown in Reaction Scheme I (FIG. 1).

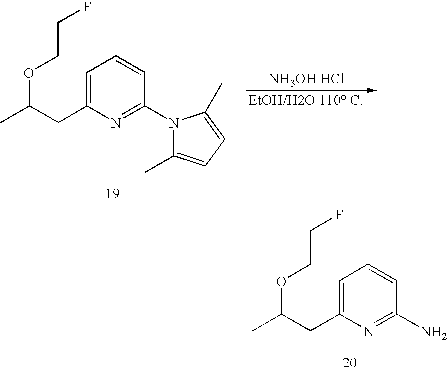

Compound 20 was synthesized from compound 19 using a standard deprotection procedure in 81% yield as liquid. The ¹H NMR spectrum (CD₃Cl, 300 MHz) of the purified product was: δ: 6.41 (s, 1H), 6.17 (s, 1H), 4.38-4.56 (m, 2H), 4.36 (s, br, 2H), 3.88 (m, 1H), 3.53-3.67 (m, 2H), 2.55-2.93 (m, 2H), 2.19 (s, 3H), 1.19 (d, 3H, J=6.3 Hz).

### Example 14

This example illustrates synthesis of compound 23, as shown in Reaction Scheme I (FIG. 1).

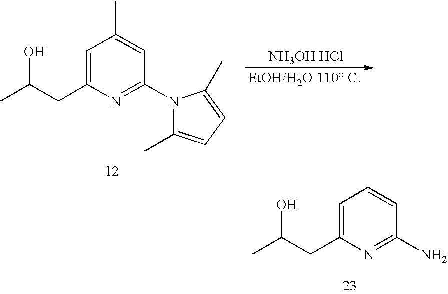

Compound 23 was synthesized from compound 12 using a standard deprotection procedure in 66% yield as slightly yellow solid (mp. 95-96° C.). The ¹H NMR spectrum (CD₃Cl, 300 MHz) of the purified product was: δ: 6.31 (s, 1H), 6.19 (s, 1H), 4.93 (s, br, 1H), 4.42 (s, br, H), 4.12 (m, 1H), 2.62 (m, 2H), 2.18 (s, 3H), 1.21 (d, 3H, J=6.3 Hz).

### Example 15

This example illustrates synthesis of compound 5, as shown in Reaction Scheme I (FIG. 1).

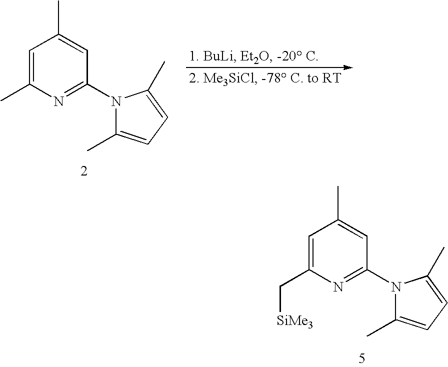

To synthesize compound 5, 4.4 ml (34.6 mmol) Me₃SiCl was added via a syringe into an Et₂O solution of lithium of compound 2 at −78 which was made from 5.78 g (28.9 mmol) of compound 2, 21.6 ml (34.6 mmol) n-BuLi (1.6 M in Hexane) in 50 ml Et₂O. The reaction mixture was allowed to warm up to room temperature and continued to stir for 30 minutes. Then, the reaction mixture was treated with Na₂CO₃ and brine, dried over NaSO₄ and the solvent was evaporated under reduced pressure. The crude product was purified with silica gel chromatograph using 1:4 EA/Hexane to afford 7.4 g (94%) of compound 5 as colorless liquid. The ¹H NMR spectrum (CD₃Cl, 300 MHz) of the purified product was: δ: 6.80 (s, 1H), 6.73 (s, 1H), 5.85 (s, 2H), 2.36 (s, 2H), 2.35 (s, 3H), 2.08 (s, 6H), 0.04 (s, 9H). Ms (ESI): m/z 273.2 (M+H⁺).

### Example 16

This example illustrates synthesis of compound 24, as shown in Reaction Scheme I (FIG. 1).

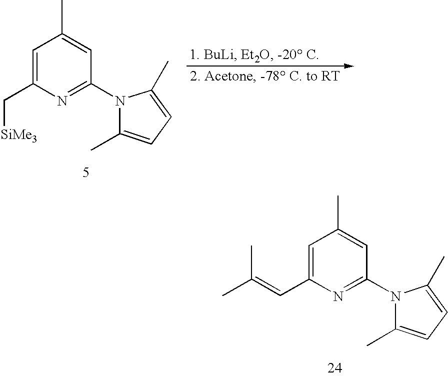

To synthesize compound 24, 2.75 ml (4.4 mmol) 1.6 M n-butyl lithium in hexane was added dropwise into a solution of 1.0 g (3.67 mmol) compound 5 in 10 ml Et₂O at −20° C. The reaction solution turned brown in color and was stirred at −20 to −10° C. for 2 hours. Then the reaction solution was cooled down to −78° C., and 2 ml acetone (treated with 0.2 m 1.6 M n-butyl lithium in hexane) was added. The solution became slightly yellow and clear, it was allowed to warm up to room temperature during 20 minutes. The solution was acidified by 6 ml 1 N HCl, followed by saturated Na₂CO₃ solution, brine, and dried over Na₂SO₄. Solvent was evaporated under reduced pressure and the crude product was purified with silica gel chromatograph using 1:15 EA/Hexane to afford 0.56 g (64%) of compound 24 as colorless liquid. The ¹H NMR spectrum (CD₃Cl, 300 MHz) of the purified product was: δ: 6.97 (s, 1H), 6.80 (s, 1H), 6.29 (s, 1H), 5.87 (s, 2H), 2.38 (s, 3H), 2.13 (s, 6H), 2.12 (s, 3H), 1.94 (s, 3H). MS (ESI): m/z 241.2 (M+H⁺).

### Example 17

This example illustrates synthesis of compound 25, as shown in Reaction Scheme I (FIG. 1).

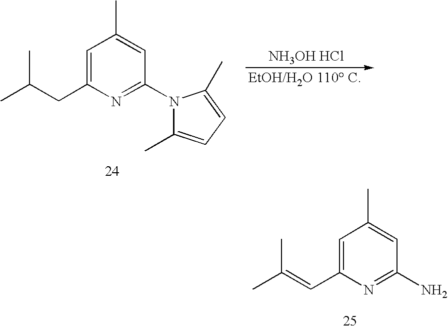

Compound 25 was synthesized from compound 24 using a standard deprotection procedure in 64% yield as slightly yellow liquid. The ¹H NMR spectrum (CD₃Cl, 300 MHz) of the purified product was: δ: 6.38 (s, 1H), 6.13 (s, 1H), 6.12 (s, 1H), 4.38 (s, br, 2H), 2.18 (s, 3H), 1.99 (s, 3H), 1.88 (s, 3H).

### Example 18

This example illustrates synthesis of compound 26, as shown in Reaction Scheme I (FIG. 1).

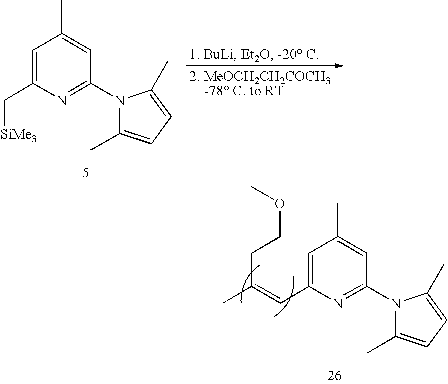

To synthesize compound 26, 0.45 g (4.4 mmol) MeOCH₂CH₂COMe, which was synthesized from methylvinylketone in MeOH using H₂SO₄ as catalyst, was added into an Et₂O solution of lithium of compound 5 at −78° C., which was made from 1.0 g (3.67 mmol) of compound 5, 2.75 ml (4.4 mmol) 1.6 M n-BuLi/Hexane in 10 ml Et₂O. The reaction mixture was allowed to warm up to room temperature during 30 minutes. Then, the reaction solution was acidified with 6 ml 1 N HCl, followed by saturated Na₂CO₃ solution, brine, and dried over Na₂SO₄. Solvent was evaporated under reduced pressure and the crude product was purified with silica gel chromatograph using 1:15 and 1:8 EA/Hexane to afford 0.25 g and 0.22 g of isomers of compound 26 as colorless liquid in 45% total yield. The ¹H NMR spectrum (CD₃Cl, 300 MHz) of the purified product was: δ: 7.01 (s, 1H), 6.81 (s, 1H), 6.35 (s, 1H), 5.85 (s, 2H), 3.55 (t, 2H, J=7.2 Hz), 3.25 (s, 3H), 2.90 (t, 2H, J=6.90 Hz), 2.38 (s, 3H), 2.11 (s, 6H), 1.97 (s, 3H) and δ: 7.00 (s, 1H), 6.80 (s, 1H), 6.33 (s, 1H), 5.87 (s, 2H), 3.58 (t, 2H, J=6.6 Hz), 3.37 (s, 3H), 2.48 (t, 2H, J=6.90 Hz), 2.38 (s, 3H), 2.16 (s, 3H), 2.13 (s, 6H).

### Example 19

This example illustrates synthesis of compound 27, as shown in Reaction Scheme I (FIG. 1).

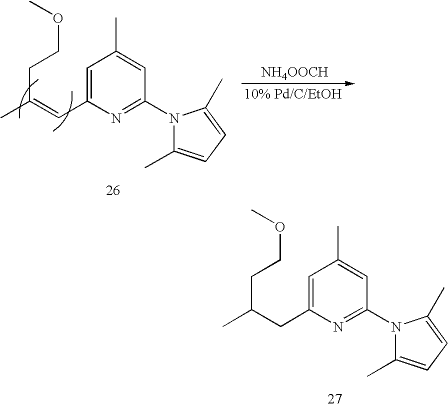

To synthesize compound 27, 0.47 g (1.7 mmol) isomer mixture of compound 26 and 20 ml absolute EtOH, followed by 0.25 g 10% Pd/C were loaded into a 100 ml round-bottom flask equipped with a big magnetic stirring bar. The reaction mixture was stirred in 80° C. oil bath, and the reaction was completed in 1 hour according to TLC. Solids were removed by filtration and solvent was evaporated under reduced pressure. The residue was dissolved in ethyl acetate and the solution was treated with 10 ml saturated Na₂CO₃ solution and brine, dried over MgSO₄. Solvent was evaporated under reduced pressure and the crude product was purified with silica gel chromatograph using 1:10 and 1:4 EA/Hexane to afford 0.38 g (81%) of compound 27 as colorless liquid. The ¹H NMR spectrum (CD₃Cl, 300 MHz) of the purified product was: δ: 6.97 (s, 1H), 6.86 (s, 1H), 5.88 (s, 2H), 3.43 (m, 2H), 3.32 (s, 3H), 2.58-2.84 (m, 2H), 2.40 (s, 3H), 2.18 (m, 1H), 2.13 (s, 6H), 1.72-1.46 (m, 2H), 0.92 (d, 3H, J=6.60 Hz).

### Example 20

This example illustrates synthesis of compound 28, as shown in Reaction Scheme I (FIG. 1).

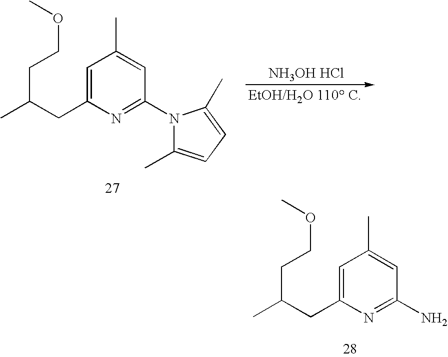

Compound 28 was synthesized from compound 27 using a standard deprotection procedure in 71% yield as slightly yellow liquid. The ¹H NMR spectrum (CD₃Cl, 300 MHz) of the purified product was: δ: 6.34 (s, 1H), 6.16 (s, 1H), 4.37 (s, br, 2H), 3.44 (m, 2H), 3.32 (s, 3H), 2.62-2.36 (m, 2H), 2.20 (s, 3H), 2.05 (m, 1H), 1.75-1.40 (m, 2H), 0.90 (d, 3H, J=6.90 Hz).

### Example 21

This example illustrates synthesis of compound 29, as shown in Reaction Scheme I (FIG. 1).

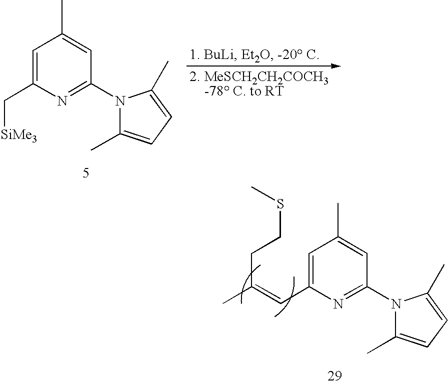

To synthesize compound 29, 0.90 g (7.61 mmol) MeSCH₂CH₂COMe was added into an Et₂O solution of lithium of compound 5 at −78° C., which was made from 1.6 g (5.87 mmol) compound 5, 4.8 ml (7.68 mmol) 1.6 M n-BuLi/Hexane in 25 ml Et₂O. The reaction mixture was allowed to warm up to room temperature during 30 minutes. Then, the reaction solution was acidified with 10 ml 1 N HCl, followed by saturated Na₂CO₃ solution, brine, and dried over Na₂SO₄. Solvent was evaporated under reduced pressure and the crude product was purified with silica gel chromatograph using 1:10 EA/Hexane to afford 1.38 g (78%) of isomers of compound 29 as colorless liquid. The ¹H NMR spectrum (CD₃Cl, 300 MHz) of the purified product was: δ: 7.00 (s, 1H), 6.95 (s, 1H), 6.80 (s, 1H), 6.73 (s, 1H), 6.33 (s, 1H), 6.29 (s, 1H), 5.87 (s, 2H), 5.83 (s, 2H), 2.95-2.48 (m, 8H), 2.39-2.35 (m, 6H), 2.16-2.09 (m, 18H), 1.96 (s, 3H), 1.88 (s, 3H).

### Example 22

This example illustrates synthesis of compound 30, as shown in Reaction Scheme I (FIG. 1).

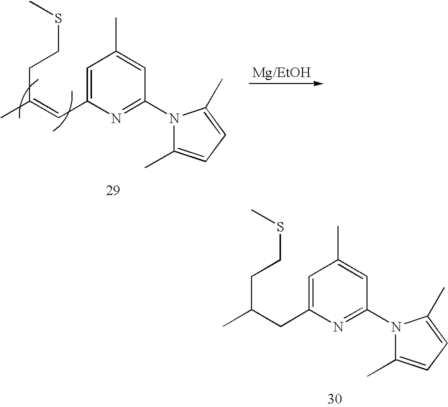

To synthesize compound 30, 1.25 g Mg turnings was added into a solution of 1.38 g (4.6 mmol) compound 29 in 30 ml EtOH at 0° C. The reaction mixture was stirred at 0° C. for 1 hour, and allowed to warm up to room temperature and stirred at room temperature overnight. All Mg turnings were consumed. The reaction mixture was acidified with 1 N HCl, and then treated with saturated Na₂CO₃ solution, extracted with 3×150 ml ethyl acetate. All the organic was combined and solvent was evaporated under reduced pressure. The residue was purified with silica gel chromatograph using 1:10 EA/Hexane to afford 0.42 g (30%) of compound 30 as colorless liquid. The ¹H NMR spectrum (CD₃Cl, 300 MHz) of the purified product was: δ: 6.98 (s, 1H), 6.86 (s, 1H), 5.88 (s, 2H), 2.50-2.80 (m, 4H), 2.40 (s, 3H), 2.15 (m, 1H), 2.12 (s, 6H), 2.07 (s, 3H), 1.7-1.5 (m, 2H), 0.92 (d, 3H, J=6.90 Hz).

### Example 23

This example illustrates synthesis of compound 31, as shown in Reaction Scheme I (FIG. 1).

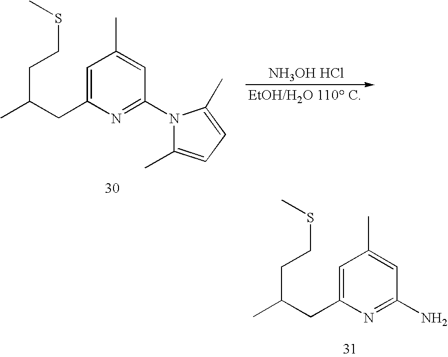

Compound 31 was synthesized from compound 30 using a standard deprotection procedure in 84% yield as slightly yellow liquid. The ¹H NMR spectrum (CD₃Cl, 300 MHz) of the purified product was: δ: 6.32 (s, 1H), 6.15 (s, 1H), 4.62 (s, br, 2H), 2.62-2.34 (m, 4H), 2.20 (s, 3H), 2.08 (s, 3H), 2.04 (m, 1H), 1.7-1.4 (m, 2H), 0.90 (d, 3H, J=6.6 Hz).

### Example 24

This example illustrates synthesis of compound 32, as shown in Reaction Scheme I (FIG. 1).

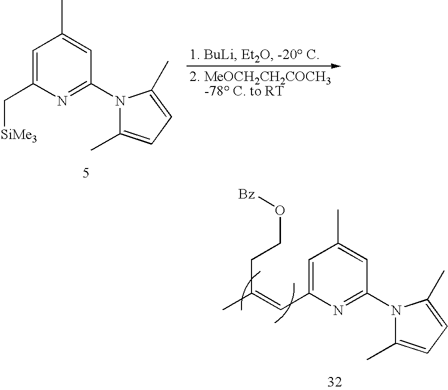

To synthesize compound 32, 3.37 g (18.9 mmol) BzOCH₂CH₂COMe was added into an Et₂O solution of lithium of compound 5 at −78° C., which was made from 4.3 g (15.8 mmol) compound 5, 11.8 ml (18.9 mmol) 1.6 M n-BuLi/Hexane in 50 ml Et₂O. The reaction mixture was allowed to warm up to room temperature during 30 minutes. Then, the reaction solution was acidified with 20 ml 1 N HCl, followed by saturated Na₂CO₃ solution, brine, and dried over Na₂SO₄. Solvent was evaporated under reduced pressure and the crude product was purified with silica gel chromatograph using 1:4 EA/Hexane to afford 4.4 g (77%) of isomers of compound 32 as colorless liquid. The ¹H NMR spectrum (CD₃Cl, 300 MHz) of the purified product was: 7.2-7.1 (m, 10H), 7.02 (s, 1H), 6.98 (s, 1H), 6.36 (s, 2H), 5.87 (s, 2H), 5.86 (s, 2H), 4.55 (s, 2H), 4.42 (S, 2H), 3.67 (t, 4H, J=4.1 Hz), 2.96 (t, 2H), 2.53 (t, 2H), 2.39 (s, 3H), 2.37 (s, 3H), 2.15 (s, 3H), 2.13 (s, 6H), 2.11 (s, 6H), 1.98 (s, 3H). IV-091.

### Example 25

This example illustrates synthesis of compound 33, as shown in Reaction Scheme I (FIG. 1).

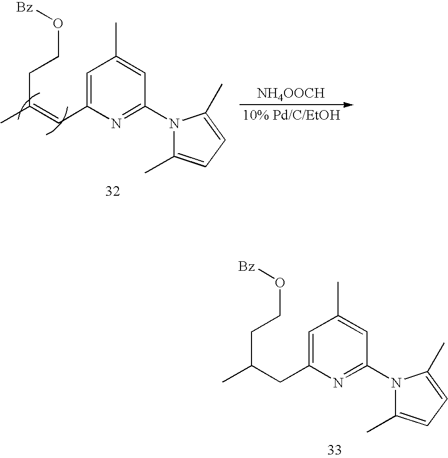

Compound 33 was synthesized using ammonium formate and 10% Pd/C in EtOH at 80° C. in 75% yield as colorless liquid. The ¹H NMR spectrum (CD₃Cl, 300 MHz) of the purified product was: δ: 7.35-7.26 (m, 5H), 6.99 (s, 1H), 6.84 (s, 1H), 5.87 (s, 2H), 4.48 (s, 2H), 3.57-3.48 (m, 2H), 2.82-2.54 (m, 2H), 2.37 (s, 3H), 2.16 (m, 1H), 2.10 (s, 6H), 1.75-1.49 (m, 2H), 0.90 (d, 3H, J=6.60 Hz).

### Example 26

This example illustrates synthesis of compound 34, as shown in Reaction Scheme I (FIG. 1).

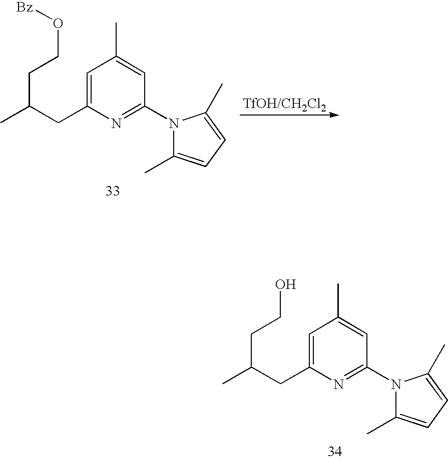

To synthesize compound 34, 4 ml TfOH was added dropwise via a glass syringe with steel needle into a solution of 2.52 g (6.9 mmol) in 20 ml CH₂Cl₂ at room temperature. The reaction mixture became warm and brown in color, and was completed in 18 minutes according to TLC. The reaction mixture was poured into 100 ml saturated NaHCO₃ solution, and extracted with 3×50 ml CH₂Cl₂. The organic solution was washed with brine, dried over Na₂SO₄. Solvent was evaporated under reduced pressure and the crude product was purified with silica gel chromatograph using 1:3 and 1:1 EA/Hexane to afford 1.5 g (82%) of compound 34 as colorless liquid. The ¹H NMR spectrum (CD₃Cl, 300 MHz) of the purified product was: δ: 6.96 (s, 1H), 6.85 (s, 1H), 5.86 (s, 2H), 3.64 (m, 2H), 2.87-2.52 (, 2H), 2.39 (s, 3H), 2.15 (m, 1H), 2.09 (s, 6H), 1.55-1.47 (m, 2H), 0.90 (d, 3H, J=6.9 Hz).

### Example 27

This example illustrates synthesis of compound 35, as shown in Reaction Scheme I (FIG. 1).

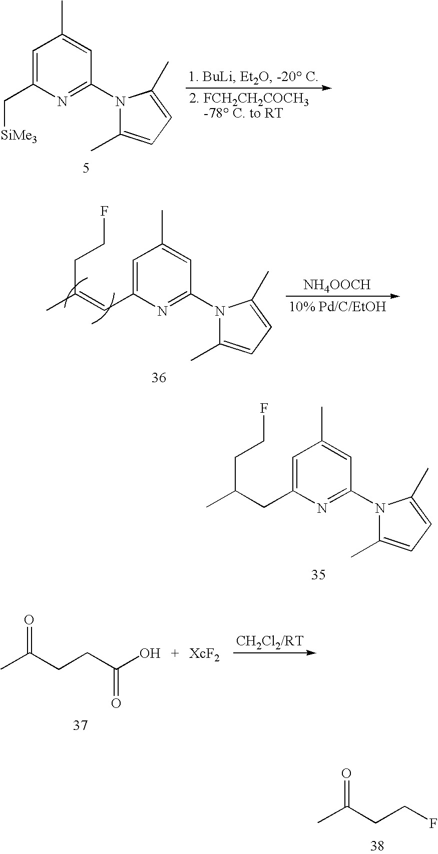

Compound 35 was synthesized by using compound 36 as an intermediate. Compound 38 was synthesized from compound 37 and XeF₂ in CH₂Cl₂. Compound 36 was synthesized as a mixture of two isomers in <50% yield. Compound 35 was reduced by ammonium formate in the presence of 10% Pd/C in 30% yield as colorless liquid. The ¹H NMR spectrum (CD₃Cl, 300 MHz) of the purified product was: δ: 6.95 (s, 1H), 6.84 (s, 1H), 5.87 (s, 2H), 4.59 (t, 1H), 4.42 (t, 1H), 2.8-2.6 (m, 2H), 2.38 (d, 2H, J=4.5 Hz), 2.2 (m, 1H), 2.11 (s, 3H), 1.9-1.5 (m, 2H), 0.95 (d, 3H, J=6.9 Hz).

### Example 28

This example illustrates synthesis of compound 39, as shown in Reaction Scheme I (FIG. 1).

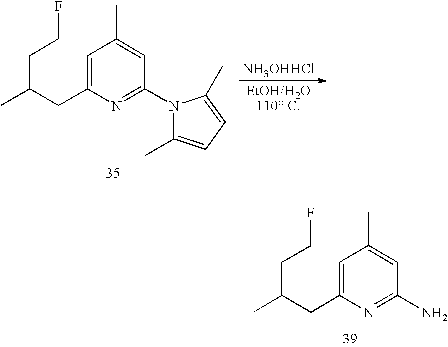

Compound 39 was synthesized from compound 36 using a standard deprotection procedure in 47% yield as slightly yellow liquid. The ¹H NMR spectrum (CD₃Cl, 300 MHz) of the purified product was: δ: 6.31 (s, 1H), 6.15 (s, 1H), 4.67 (s, br, 2H), 4.50 (dt, 2H, J=47.4, 6.3 Hz), 2.6-2.4 (m, 2H), 2.19 (s, 3H), 2.1-2.0 (m, 1H), 1.8-1.5 (m, 2H), 0.93 (d, 3H, J=6.3 Hz). ¹⁹F NMR (CD₃Cl, 282.2 MHz) δ: −41.4. Compound 39 oxalic acid results in a white solid with a melting point of 136.6-137.1° C.

### Example 29

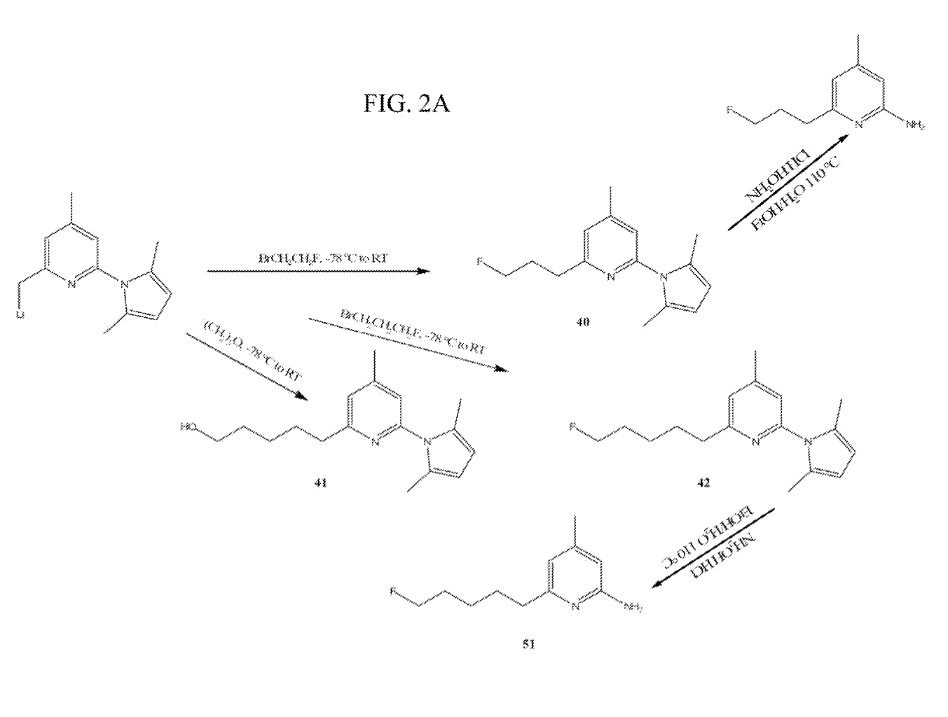

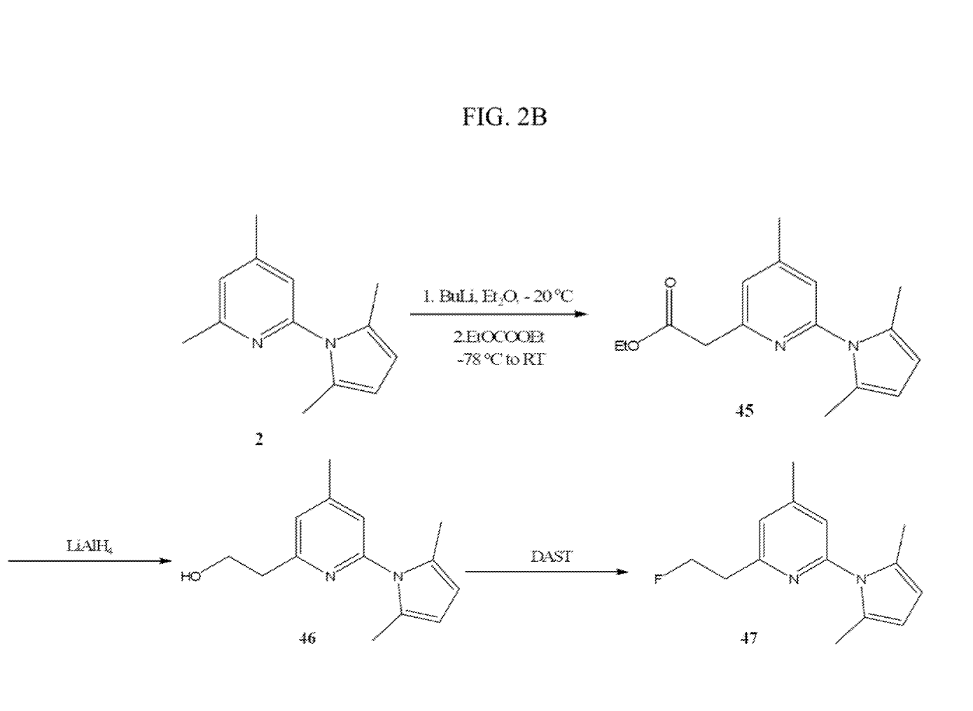

This example illustrates synthesis of compound 40, as shown in Reaction Scheme II (FIG. 2).

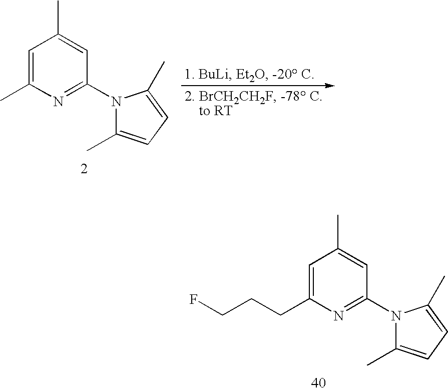

To synthesize compound 40, 2 g (15.7 mmol) BrCH₂CH₂F was added via a syringe into an Et₂O solution of lithium of compound 2 at −78° C., which was made from 2.0 g (10 mmol) compound 2, 8.0 ml (12.8 mmol) n-BuLi (1.6 M in Hexane) in 20 ml Et₂O. The reaction mixture was allowed to warm up to room temperature and continued to stir for 30 minutes. Then, the reaction mixture was treated with Na₂CO₃ and brine, dried over Na₂SO4 and the solvent was evaporated under reduced pressure. The crude product was purified with silica gel chromatograph using 1:8 EA/Hexane to afford 1.6 g (61%) of compound 40 as colorless liquid. ¹H NMR (CD₃Cl, 300 MHz) δ: 7.04 (s, 1H), 6.91 (s, 1H), 5.92 (s, 2H), 4.53 (dt, 2H, J=47.4, 6.0 Hz), 2.95 (t, 2H, J=7.5 Hz), 2.44 (s, 3H), 2.2-2.1 (m, 2H), 2.16 (s, 6H). ¹⁹F NMR (CD₃Cl, 282.2 MHz) δ: −42.9.

### Example 30

This example illustrates synthesis of compound 41, as shown in Reaction Scheme II (FIG. 2).

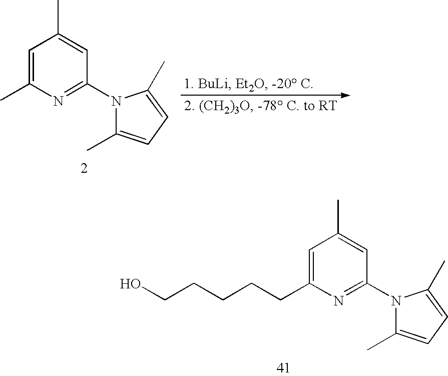

To synthesize compound 41, 0.42 ml (6.4 mmol) (CH₂)₃O was added via a syringe into an Et₂O solution of lithium of compound 2 at −78° C., which was made from 1.0 g (5 mmol) compound 2, 4.0 ml (6.4 mmol) n-BuLi (1.6 M in Hexane) in 10 ml Et₂O. The reaction mixture was allowed to warm up to room temperature and continued to stir for 30 minutes. Then, the reaction mixture was treated with NaHCO₃ and brine, dried over Na₂SO4 and the solvent was evaporated under reduced pressure. The crude product was purified with silica gel chromatograph using 1:1 EA/Hexane to afford 0.76 g (74%) of compound 41 as colorless liquid. The ¹H NMR spectrum (CD₃Cl, 300 MHz) of the purified product was: δ: 6.98 (s, 1H), 6.85 (s, 1H), 5.87 (s, 2H), 3.65 (t, 2H, J=6.4 Hz), 2.81 (t, 2H, J=7.5 Hz), 2.39 (s, 3H), 2.11 (s, 6H), 1.8-1.6 (m, 4H).

### Example 31

This example illustrates synthesis of compound 42, as shown in Reaction Scheme II (FIG. 2).

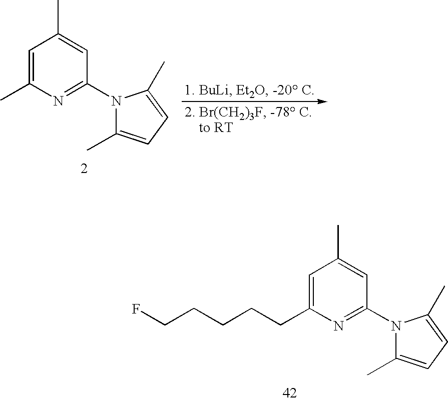

To synthesize compound 42, 2.1 g (15.0 mmol) BrCH₂CH₂CH₂F was added via a syringe into an Et₂O solution of lithium of compound 2 at −78° C., which was made from 3.0 g (15 mmol) compound 2, 11.0 ml (17.6 mmol) n-BuLi (1.6 M in Hexane) in 30 ml Et₂O. The reaction mixture was allowed to warm up to room temperature and continued to stir for 30 minutes. Then, the reaction mixture was treated with NaHCO₃ and brine, dried over Na₂SO4 and the solvent was evaporated under reduced pressure. The crude product was purified with silica gel chromatograph using 1:10 EA/Hexane to afford 2.5 g (63%) of compound 42 as colorless liquid. The ¹H NMR spectrum (CD₃Cl, 300 MHz) of the purified product was: δ: 6.98 (s, 1H), 6.85 (s, 1H), 5.87 (s, 2H), 4.55 (t, 1H, J=5.9 Hz), 4.39 (t, 1H, J=5.9 Hz), 2.82 (t, 2H, 7.5 Hz), 2.39 (s, 3H), 2.11 (s, 6H), 1.9-1.7 (m, 4H). The ¹⁹F NMR spectrum (CD₃Cl, 282.2 MHz) of the purified product was δ: −41.7.

### Example 32

This example illustrates synthesis of compound 44.

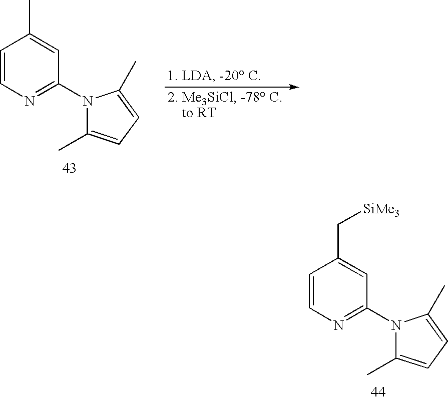

To synthesize compound 44, 0.91 ml (6.44 mmol) (iPr)₂NH was added into a solution of 1.0 g (5.36 mmol) compound 43 in 20 ml dry Et₂O. The reaction mixture was cooled down to −20 and 4.0 ml (6.4 mmol) 1.6 M n-BuLi in Hexane was added. After a few minutes, yellowish white solids precipitated from the solution, and the reaction was completed in 30 minutes high amount of solids formed in the reaction mixture. After the mixture was cooled down to −78° C., 0.81 g (6.4 mmol) Me₃SiCl was added, and the reaction mixture was allowed to warm up to room temperature, and continued to stir for one hour. The reaction mixture was treated with saturated NaHCO₃, diluted with ethyl acetate, washed with brine, and the organic was dried over MgSO₄. The solvent was evaporated under reduced pressure, and the crude product was purified with silica gel chromatograph using 1:4 EA/Hexane to afford 1.18 g (85%) of compound 44 as colorless liquid. The ¹H NMR spectrum (CD₃Cl, 300 MHz) of the purified product was: δ: 8.40 (d, 1H, J=5.1 Hz), 6.95 (d, 1H, J=5.1 Hz), 6.87 (s, 1H), 5.89 (s, 2H), 2.20 (s, 2H), 2.12 (s, 6H), 0.62 (s, 9H).

### Example 33

This example illustrates synthesis of compound 45, as shown in Reaction Scheme II (FIG. 2).

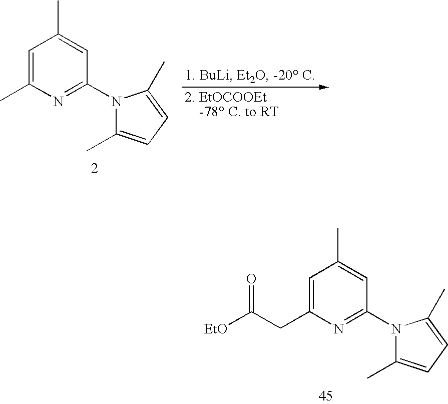

To synthesize compound 45, a solution of Et₂O solution of lithium of compound 2 at −78° C., which was made from 5.0 g (25 mmol) compound 2, 18 ml (28.8 mmol) n-BuLi (1.6 M in Hexane) in 100 ml Et₂O was added via a cannula into a solution of 10 ml EtOCOOEt in 50 ml Et₂O at −78° C. The reaction mixture was allowed to warm up to room temperature during 30 minutes. Then, the reaction mixture was diluted with ethyl acetate, washed with saturated NaHCO₃ solution, brine and the organic was dried over Na₂SO₄. The solvent was evaporated under reduced pressure. The crude product was purified with silica gel chromatograph using 1:8 EA/Hexane to afford 2.7 g (40%) of compound 45 as colorless liquid and 2.0 g of compound 2. The ¹H NMR spectrum (CD₃Cl, 300 MHz) of the purified product was: δ: 7.12 (s, 1H), 6.93 (s, 1H), 5.87 (s, 2H), 4.17 (q, 2H, J=6.3 Hz), 3.82 (s, 2H), 2.42 (s, 3H), 2.11 (s, 6H), 1.25 (t, 3H, J=6.3 Hz).

### Example 34

This example illustrates synthesis of compound 46.

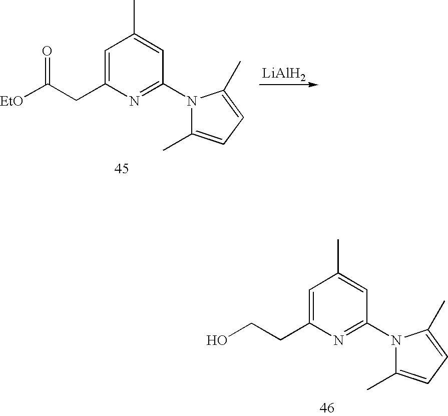

To synthesize compound 46, 10 ml 1 M LiAlH₄ in ether was added dropwise into a solution of 2.7 g (10 mmol) compound 45 in 40 ml THF. Upon the completion of the reaction mixture in 4 hours according to TLC, saturated ammonium chloride solution was added. The organic solution was separated and washed by saturated NaHCO₃, brine and dried over MgSO₄. The solvent was evaporated under reduced pressure. The crude product was purified with silica gel chromatograph using 1:1 EA/Hexane to afford 1.7 g (75%) of compound 46 as colorless liquid. The ¹H NMR spectrum (CD₃Cl, 300 MHz) of the purified product was: δ: 6.99 (s, 1H), 6.89 (s, 1H), 5.88 (s, 2H), 4.01 (t, 2H, J=5.1 Hz), 3.80 (s, br, 1H), 3.02 (t, 2H, J=5.1 Hz), 2.40 (s, 3H), 2.13 (s, 6H).

### Example 35

This example illustrates synthesis of compound 47, as shown in Reaction Scheme II (FIG. 2).

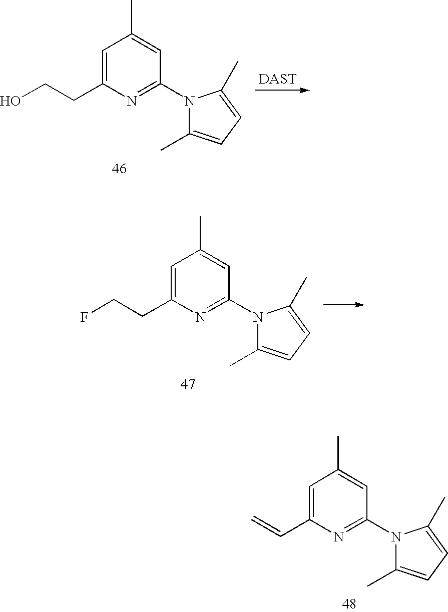

To synthesize compound 47, 0.32 ml (2.46 mmol) DAST was added into a solution of 0.38 g (1.65 mmol) compound 46 in 10 ml CH₂Cl₂ at 0° C. After 1 hour at 0° C., more than 90% of compound 46 reacted, and compound 47 is the major product on TLC. The reaction mixture was quenched with 10 ml saturated NaHCO₃ solution, and extracted with CH₂Cl₂. The organic solution was washed with brine and dried over MgSO₄. Solvent was evaporated under reduced pressure, and the residue was submitted for silica gel purification using 1:4 EA/Hexane. However, it was found that compound 47 may decompose on silica gel to form compound 48. Compound 47 was confirmed by ¹HMR. The ¹H NMR spectrum (CD₃Cl, 300 MHz) of the purified product was: δ: 7.05 (s, 1H), 6.91 (s, 1H), 5.89 (s, 2H), 4.84 (dt, 2H, J=46.8, 6.0 Hz), 3.17 (dt, 2H, J=25.8, 6.0 Hz), 2.41 (s, 3H), 2.13 (s, 6H).

### Example 36

This example illustrates synthesis of compound 49, as shown in Reaction Scheme II (FIG. 2).

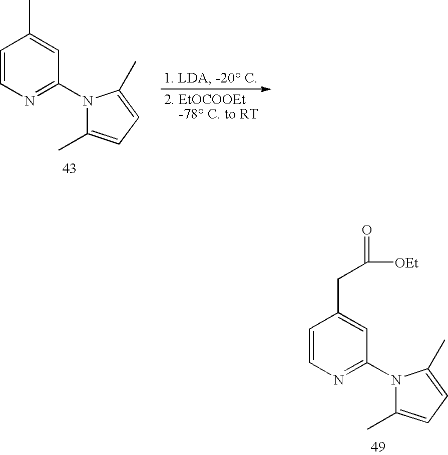

To synthesize compound 49, a solution of Et₂O solution of lithium of compound 43 at −78° C., which was made from 5.0 g (25 mmol) compound 43, 4.55 ml (32 mmol) (i-Pr)₂NH and 20 ml (32 mmol) n-BuLi (1.6 M in Hexane) in 80 ml Et₂O was added via a cannula into a solution of 10 ml EtOCOOEt in 50 ml Et₂O at −78° C. The reaction mixture was allowed to warm up to room temperature during 30 minutes. Then, the reaction mixture was diluted with ethyl acetate, washed with saturated NaHCO₃ solution, brine and the organic was dried over Na₂SO₄. The solvent was evaporated under reduced pressure. The crude product was purified with silica gel chromatograph using 1:8 EA/Hexane to afford 2.8 g (40%) of compound 49 as colorless liquid and 1.75 g of compound 43. The ¹H NMR spectrum (CD₃Cl, 300 MHz) of the purified product was: δ: 8.58 (d, 1H, J=4.8 Hz), 7.21 (d, 1H, J=4.8 Hz), 7.19 (s, 1H), 5.89 (s, 2H), 4.21 (m, 2H), 3.68 (s, 2H), 2.13 (s, 6H), 1.25 (t, 3H).

### Example 37

This example illustrates synthesis of compound 50, as shown in Reaction Scheme II (FIG. 2).

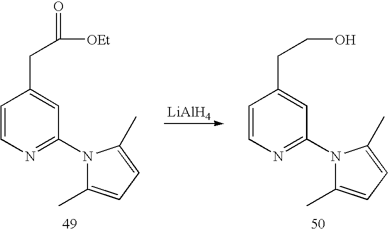

To synthesize compound 50, 10 ml 1 M LiAlH₄ in ether was added dropwise into a solution of 2.8 g (10.8 mmol) compound 49 in 40 ml THF. Upon the completion of the reaction mixture in 4 hours according to TLC, saturated ammonium chloride solution was added. The organic solution was separated and washed by saturated NaHCO₃, brine and dried over MgSO₄. The solvent was evaporated under reduced pressure. The crude product was purified with silica gel chromatograph using 1:4 and 1:1 EA/Hexane to afford 0.64 g (27%) of compound 50 as solids with slightly yellow color. The ¹H NMR spectrum (CD₃Cl, 300 MHz) of the purified product was: δ: 8.49 (d, 1H, J=5.1 Hz), 7.19 (d, 1H, J'25.1 Hz), 7.11 (s, 1H), 5.89 (s, 2H), 3.93 (t, 2H, J=6.3 Hz), 2.93 (t, 2H, 6.3 Hz), 2.12 (s, 6H).

### Example 38

This example illustrates synthesis of compound 51, as shown in Reaction Scheme II (FIG. 2).

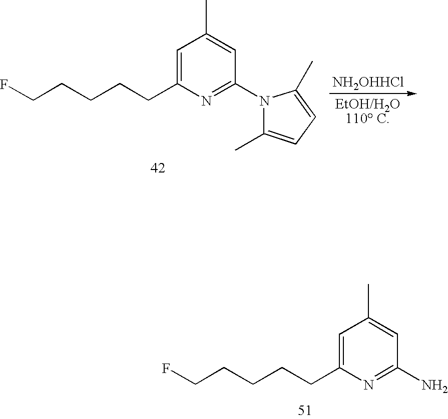

Compound 51 was synthesized from compound 42 using a standard deprotection method. The ¹HMR spectrum (CD₃Cl, 300 MHz) of the purified product was: δ: 6.34 (s, 1H), 6.15 (s, 1H), 4.53 (t, 1H), 4.39 (s, br, 1H), 4.37 (t, 1H), 2.59 (t, 2H, J=7.4 Hz), 2.18 (s, 3H), 1.8-1.65 (m, 4H). ¹⁹F NMR (CD₃Cl, 282.2 MHz) δ: −41.5.

### Example 39

This example illustrated synthesis of compound 52, as shown in Reaction Scheme II (FIG. 2).

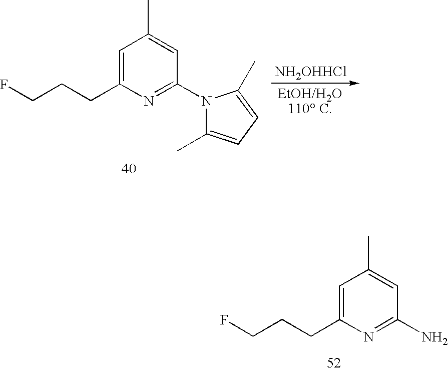

Compound 52 was synthesized from compound 40 using a standard deprotection method.

### Example 40

This example illustrates a general method for selecting compounds for labeling with ¹⁸F. The IC₅₀ for inhibiting iNOS was determined for various compounds, as shown in FIG. 3 and FIG. 4 In these experiments, IC₅₀ determinations for a compound was made by measuring iNOS activity in the presence of a compound at multiple concentrations. iNOS activity was measured by assays using the nitric oxide synthase screening kit (GE Healthcare Biosciences Corp., Piscataway, N.J.) following the manufacturer's protocols. Standards for these experiments are shown in Table 1. IC₅₀ determinations are shown in Table 2. We report our IC₅₀ determinations using the compounds S-ethylisothiourea (SEITU), L-N⁶-iminoethyl-lysine (L-NIL) and 2-aminopurine (2-AP) as standards.

TABLE 1
Standards for measuring IC₅₀

| Compound | IC₅₀ (nM) | Reported |
|---|---|---|
| SEITU | 30.5 ± 4.6 | Kᵢ = 32 nM |
| L-NIL | 1465 ± 148 | IC₅₀ = 1400 nM |
| 2-AP | 108.2 ± 34.5 | IC₅₀ = 170 nM |

TABLE 2
IC₅₀ of tested compounds

| Compound | Figure | IC₅₀ (nM) |
|---|---|---|
| DZ(III)-70/Compound 11 | 3A | 193.2 ± 38* |
| DZ(III)-73 | 3A | 282.4 ± 48.6 |
| DZ(III)-74/Compound 14 | 3A | 54.3 ± 8.9 |
| DZ(III)-85/Compound 25 | 3A | 685 ± 127 |
| DZ(III)-77/Compound 14 | 3B | 1776 ± 395 |
| DZ(III)-82/Compound 16 | 3B | >5000 |
| DZ(III)-83/Compound 28 | 3B | >5000 |
| DZ(III)-84/Compound 20 | 3B | >5000 |
| DZ(III)-86/Compound 31 | 3B | >5000 |
| DZ(IV)-93 | 4A | 731 ± 87 |
| DZ(IV)-104 | 4A | 57.6 ± 5.3 |
| DZ(IV)-120 | 4A | 170.2 ± 26.5 |
| DZ(V)-80 | 4B | 1169 ± 391 |
| DZ(V)-81 | 4B | 789 ± 111** |
| DZ(V)-82 | 4B | 800 ± 82 |
| DZ(V)-83 | 4B | >5000 |

*Reported IC₅₀ = 28 nM (Hagmann, W. K., et al., Bioorg. Med. Chem. Lett. 10: 1975-1978, 2000).
**Reported IC₅₀ = 53 nM (Connolly, S., et al., J. Med. Chem. 47: 3320-3323, 2004).

### Example 41

This example describes data for the selectivity of two candidate iNOS inhibitory compounds, as shown in FIG. 5 and Table 3. All assays were performed using the nitric oxide synthase screening kit (GE Healthcare Biosciences Corp., Piscataway, N.J.) following the manufacturer's protocols.

TABLE 3
Selectivity (IC₅₀) measured in nM

| Compound | iNOS | nNOS | eNOS |
|---|---|---|---|
| DZ(III)-74/Compound 14 | 54.3 ± 8.9 | 493.2 ± 80.8 | 1518 ± 300 |
| DZ(IV)-104 | 57.6 ± 5.3 | 514.3 ± 83.3 | 1428 ± 158 |

### Example 42

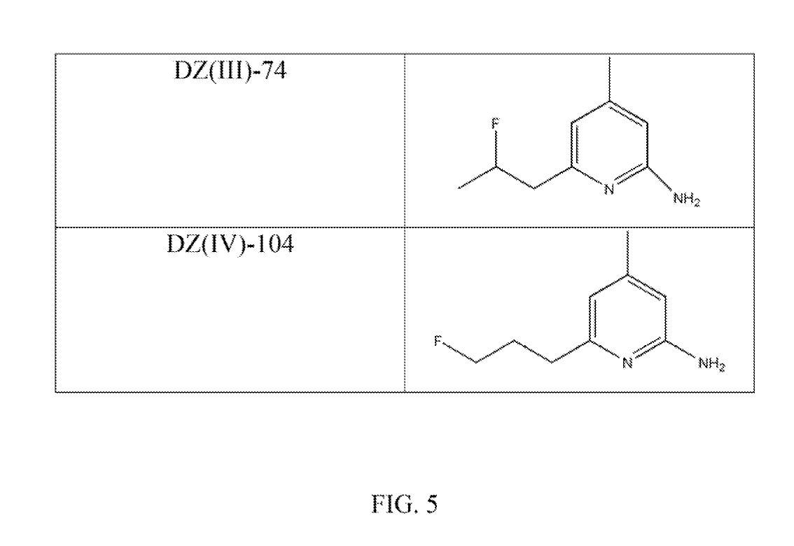

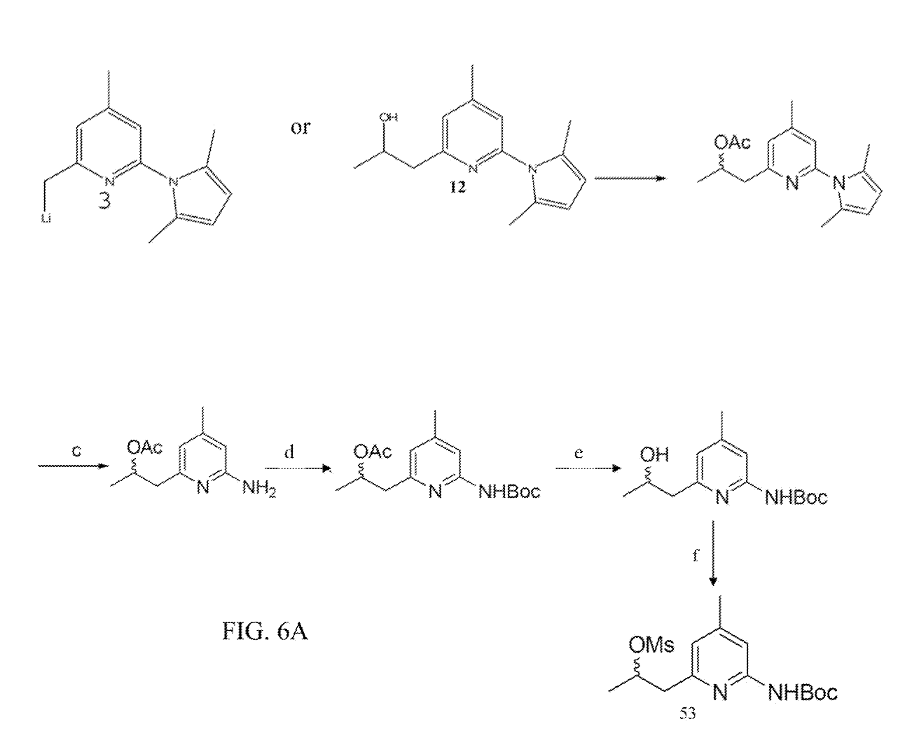

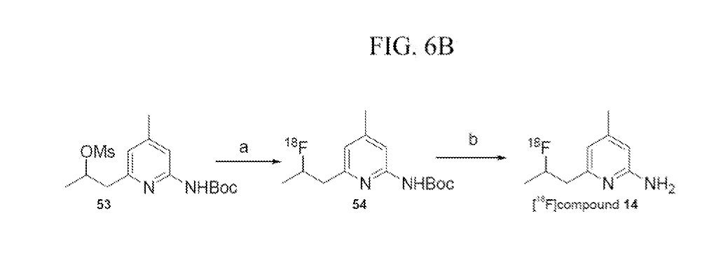

This example illustrates a general method for labeling an iNOS inhibitor with ¹⁸F, such as ¹⁸F labeling of a 2-amino-4-alkyl-6-(haloalkyl)pyridine compound, in particular [¹⁸F]compound 14, as shown in Reaction Scheme III (FIG. 6). For this synthesis, a mesylate precursor, compound 53, was synthesized as shown in FIG. 6A. As shown in FIG. 6A, synthesis of compound 53 included mixing CH₃CHO, Et₂O, −78° C. to RT; then AcOEt, Et₂O to yield compound 6; (b) AcCl, Et₃N, CH₂Cl₂ then yielded compound 7; (c) NH₂OH.HCl, EtOH, H₂O, followed by (d) Boc₂O, t-BuOH; then (e) K₂CO₃, MeOH, H₂O; and finally (f) MsCl, Et₃N, CH₂Cl₂ to yield compound 53. As shown in FIG. 6B, Compound 53 was then treated with [¹⁸F]fluoride/potassium carbonate and 4,7,13,16,21,24-Hexaoxa-1,10-diazabicyclo[8.8.8]-hexacosane (Kryptofix 222®, Acros Organics N.V., Fairlawn, N.J.) using acetonitrile (MeCN) as the solvent, 110° C./5-10 min. Further reaction with 1 N HCl and irradiation in a microwave oven for 30 seconds produced [¹⁸F]compound 14. Analysis by thin layer chromatography indicated the yield of [¹⁸F]compound 14 was 10-20%. The specific activity was 1000 Ci/mmol. In another method, reaction between compound 53, [¹⁸F], 4,7,13,16,21,24-Hexaoxa-1,10-diazabicyclo[8.8.8]-hexacosane, Cs₂CO₃ using acetonitrile (MeCN) as the solvent and heating to 110° C. for 10 min. produced [¹⁸F]compound 14 at a yield of 40% as determined by TLC.

### Example 43

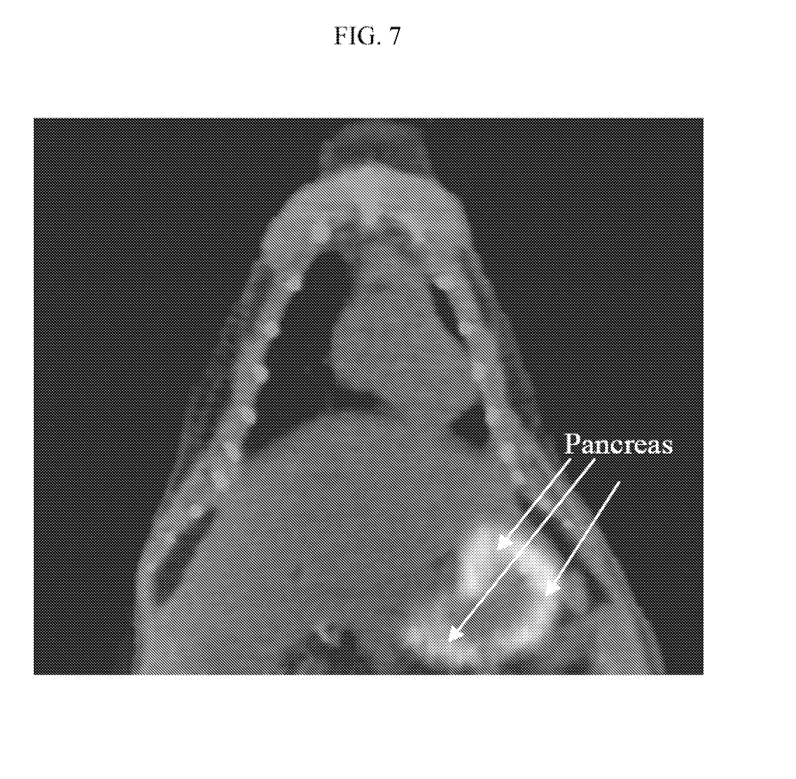

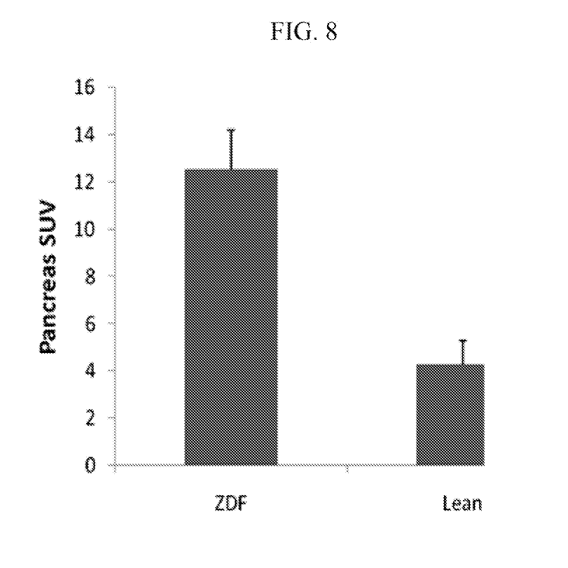

This example illustrates imaging of a Zucker Diabetic Fatty (ZDF) rat's pancreas in vivo using [¹⁸F]compound 14 as the radio tracer. Small Animal PET/CT with [¹⁸F]compound 14 was used in these experiments. The ZDF rat is an animal model of type 2 diabetes (Schmidt, R. E., et al. Am J Pathol 163: 21-28, 2003). iNOS is increased by about 20 fold in the pancreas of ZDF rats compared to controls (Shimabukuro, M., et al, J Clin Invest. 100(2): 290-295, 1997). A PET/CT image of the rat's pancreas was taken 60 minutes post injection of the [¹⁸F]compound 14 radio tracer. FIG. 7 shows a qualitative image of fused microPET/CT of male ZDF rat. FIG. 8 shows ratio of tracer uptake at 60 min. post injection. Note the contrast in the image showing iNOS distribution in the ZDF rat's pancreas (FIG. 7) and the standard uptake value (SUV) of the radio tracer in the pancreas of the ZDF rat as compared to the Lean rat, as shown in FIG. 8.

### Example 44

This example illustrates, in FIG. 9, imaging of a male Wistar rat's pancreas and liver in vivo using [¹⁸F]compound 14 as a radio tracer. In these experiments, one rat was injected with Streptozotocin (STZ) to induce Type 1 diabetes; a sham treated rat served as a control. MicroPET images of the rats were acquired at 3 weeks and at 8 weeks post injection. FIG. 9 illustrates representative coronal microPET images of [¹⁸F]compound 14 showing uptake in liver and pancreas. The images indicate iNOS concentrated in the pancreas of the rat which received the STZ treatment. The data from this MicroPET experiment is presented in graphical form in FIG. 10.

### Example 45

This example demonstrates that [¹⁸F]compound 14 is stable in rat blood. The in vitro stability of [¹⁸F]compound 14 was tested by using heparinized blood from an adult male Sprague-Dawley rat. [¹⁸F]compound 14 was relatively stable in the whole blood for 2 h at 37° C. At 1 h, approximately 80% of the activity recovered from lysed whole blood was observed as [¹⁸F]compound 14, and at 2 h, approximately 75% of the recovered activity was still in the form of [¹⁸F]compound 14. These data suggest that the blood is not a major site for compound degradation, which is in contrast to the previously reported ¹⁸F and ¹¹C labeled isothiourea compounds.

### Example 46

This example demonstrates that [¹⁸F]compound 14 is reasonably stable in vivo on Sprague-Dawley rats. Adult male Sprague-Dawley rats were injected with [¹⁸F]compound 14, and at 5 and 30 minutes post injection, plasma samples were obtained. Supernatants from the plasma samples were analyzed by silica gel radio-TLC and reversed phase HPLC. After 30 minutes, the percent parent compound was only 20.2%. Additionally, within 5 minutes post injection, only 40.3% of the activity in the blood was [¹⁸F]compound 14, which was confirmed by HPLC co-elution with non-radioactive compound 14. According to the HPLC analysis, the major metabolite, constituting 50% of the activity in blood at 5 minutes post injection, was very polar but was not free [¹⁸F] fluoride. This observation is also consistent with the low bone uptake reported in biodistribution studies. Since [¹⁸F]compound 14 demonstrated reasonable stability in whole blood, the metabolite observed in vivo must be due to metabolism in peripheral organs.

### Example 47

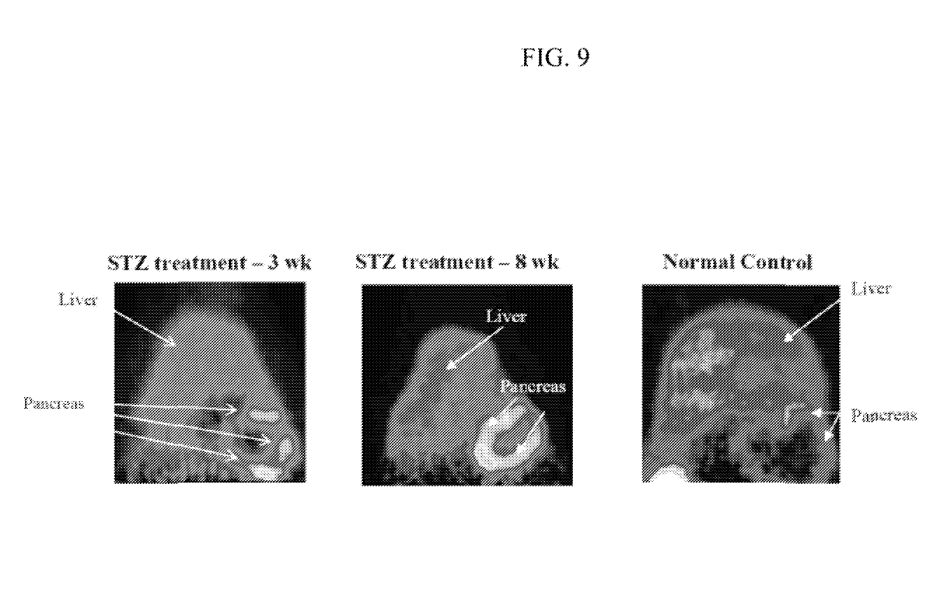

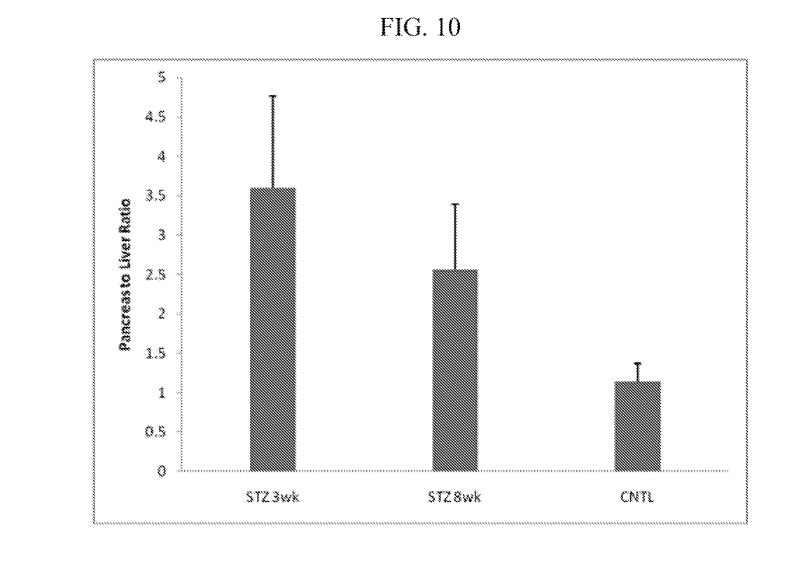

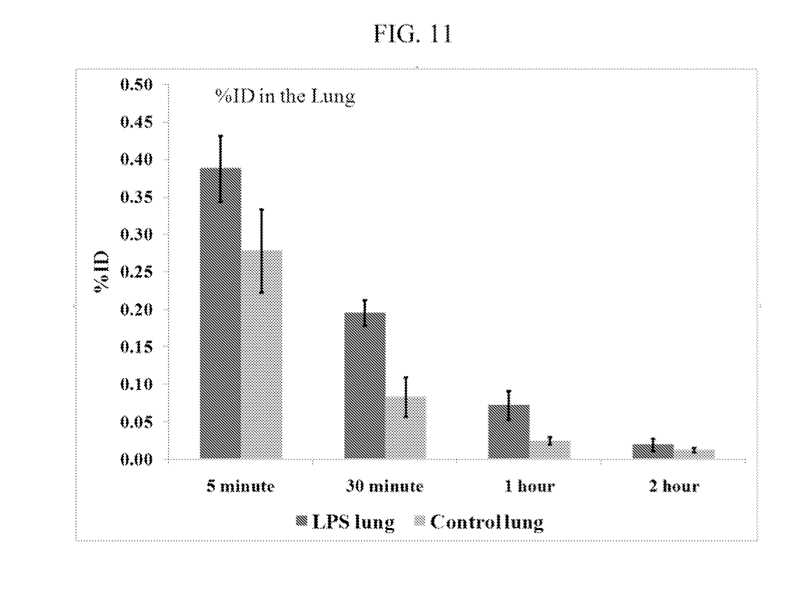

This example describes the results of in vivo studies on the biodistribution of iNOS inhibitors. The biodistribution studies were performed in mature male C57BL/6 mice. One group of mice was treated with bacterial lipopolysaccharide (LPS) (10 mg/kg, administered intravenously) to induce iNOS expression, and a control group was left untreated. LPS is a well-documented inducer of iNOS expression in both rats and mice; administration of LPS resulted in elevated iNOS expression at 6-7 h post LPS-treatment. Table 4 and FIG. 11 show that LPS-treated mice have a greater uptake of [¹⁸F]compound 14 than non-treated mice. This result demonstrates that [¹⁸F]compound 14 is a suitable PET tracer for iNOS, especially in comparison to previously described ¹⁸F-labeled isothiourea analogs.

TABLE 4
Biodistribution of [¹⁸F]9 in control vs. LPS treated mice^{a}
(data reported as mean % ID/g ± SD; n = 4)

| | 5 min | | 30 min | |
|---|---|---|---|---|
| % ID/g | LPS | Control | LPS | Control |
| Blood | 3.69 ± 0.38 | 2.37 ± 0.18 | 2.09 ± 0.35 | 1.22 ± 0.16 |
| Lung | 4.32 ± 0.32 | 3.33 ± 0.28 | 1.84 ± 0.27 | 1.14 ± 0.13 |
| Liver | 24.6 ± 3.1 | 33.2 ± 3.3 | 7.06 ± 0.28 | 10.7 ± 0.3 |
| Kidney | 26.7 ± 4.1 | 20.6 ± 2.4 | 10.9 ± 2.8 | 7.30 ± 0.66 |
| Muscle | 1.64 ± 0.09 | 1.25 ± 0.06 | 1.06 ± 0.40 | 0.71 ± 0.31 |
| Heart | 2.03 ± 0.06 | 1.50 ± 0.12 | 1.02 ± 0.16 | 0.60 ± 0.05 |
| Brain | 1.81 ± 0.09 | 1.60 ± 0.17 | 0.52 ± 0.15 | 0.28 ± 0.02 |
| Bone | 1.59 ± 0.15 | 1.52 ± 0.36 | 3.70 ± 1.20 | 2.26 ± 0.55 |
| | 1 h | | 2 h | |
| % ID/g | LPS | Control | LPS | Control |
| Blood | 0.86 ± 0.09 | 0.31 ± 0.04 | 0.23 ± 0.05 | 0.27 ± 0.22 |
| Lung | 0.73 ± 0.17 | 0.31 ± 0.06 | 0.18 ± 0.09 | 0.15 ± 0.03 |
| Liver | 2.63 ± 0.52 | 2.59 ± 0.49 | 0.42 ± 0.15 | 0.74 ± 0.23 |
| Kidney | 3.95 ± 0.53 | 2.20 ± 0.25 | 0.91 ± 0.63 | 0.80 ± 0.27 |
| Muscle | 0.78 ± 0.47 | 0.27 ± 0.18 | 0.23 ± 0.13 | 0.34 ± 0.27 |
| Heart | 0.41 ± 0.10 | 0.19 ± 0.02 | 0.12 ± 0.04 | 0.08 ± 0.02 |
| Brain | 0.19 ± 0.03 | 0.11 ± 0.02 | 0.07 ± 0.03 | 0.07 ± 0.03 |
| Bone | 4.30 ± 0.90 | 2.57 ± 0.64 | 3.77 ± 1.41 | 5.59 ± 4.20 |

^{a}LPS mice were treated with lipopolysaccharide (LPS) 10 mg/kg i.v. 6 h prior to tracer injection; all mice were injected with 50 μCi/110 μL [¹⁸F]9;
^{b}SD: standard deviation.

### Example 48

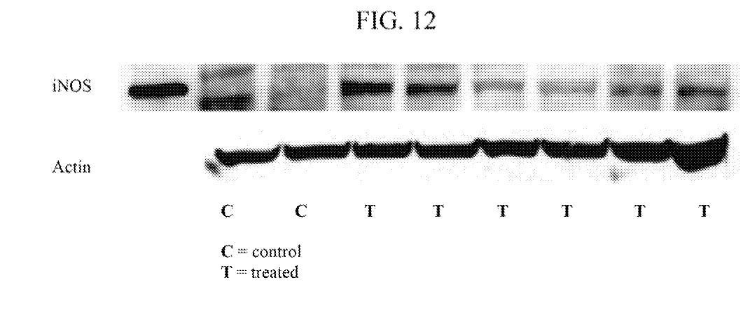

This example demonstrates that iNOS protein accumulates in the lungs of LPS-treated mice. A standard Western blot was performed to compare iNOS induction in the LPS-treated and control mice described in the preceeding Example. FIG. 12 shows the blot. The first lane of the blot shows purified iNOS as a control. The lanes labeled “C” contain protein samples from untreated mice. The lanes labeled “T” contain protein samples from LPS-treated mice. The results of the Western blot are consistent with the increased uptake of [¹⁸F]compound 14 in the LPS-treated mice (Table 1, FIG. 11).

### Example 49

This example describes blocking studies. A non-selective NOS inhibitor, 2-amino-4-methylpyridine, administered intravenously at 10 mg/kg failed to block increased uptake of [¹⁸F]compound 14 in LPS-treated mice.

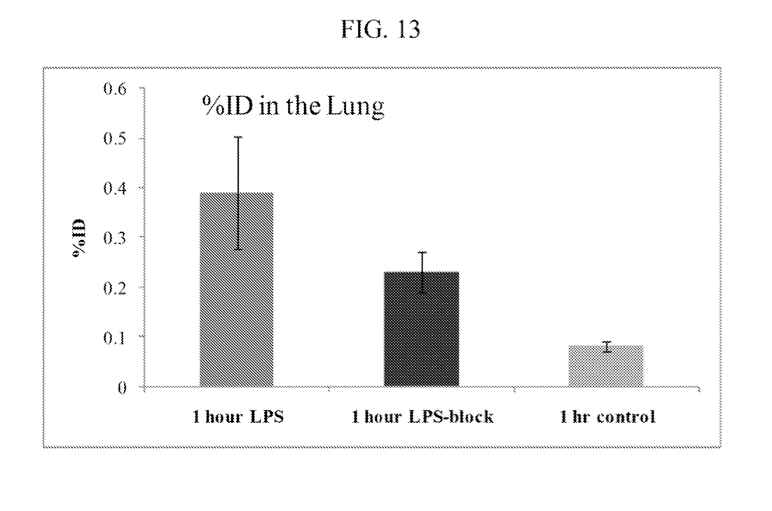

A highly selective iNOS inhibitor was then used as an iNOS inhibitor. N-(3-(aminomethyl)benzyl)acetamidine (1400 W) is a slow, tight binding, and highly selective inhibitor of iNOS in vitro and in vivo (Garvey, E. P., et al., J. Biol. Chem. 272: 4959-4963, 1997). In these experiments, 5 mg/kg 1400 W was injected intravenously immediately before the injection of [¹⁸F]compound 14. At 1 h post injection, % ID in lung was determined. As shown in FIG. 13, 32% of the tracer uptake in the lungs and blood of the LPS-treated mice was blocked. This result shows that the uptake of [¹⁸F]compound 14 in the LPS-treated mice is iNOS-specific.

### Example 50

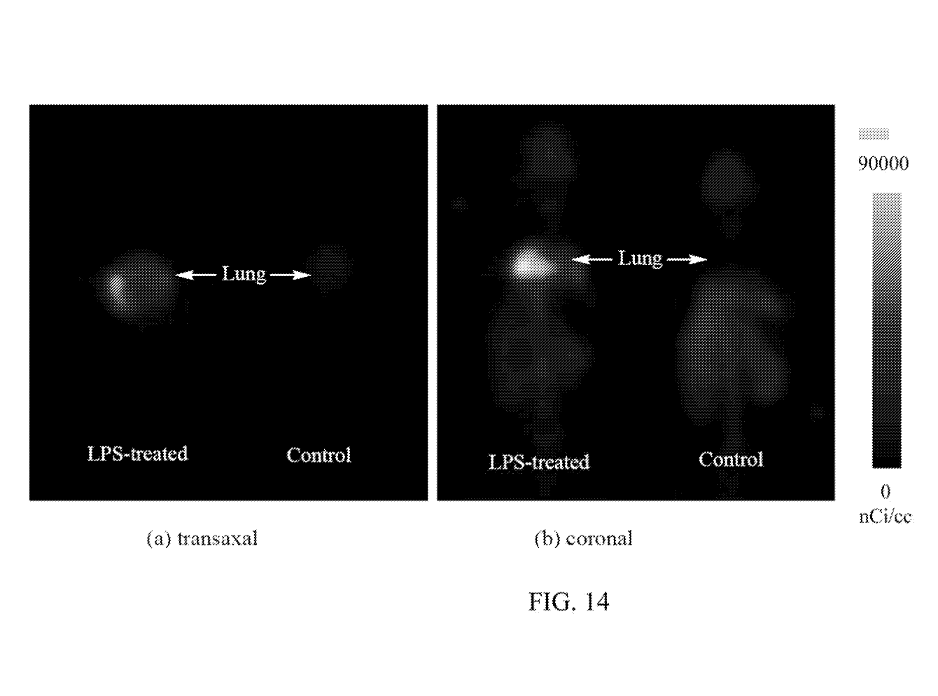

This example shows the results of a microPET imaging study using [¹⁸F]compound 14. Two mice, one intratracheally treated with LPS, and a control mouse, were injected with [¹⁸F]compound 14. FIG. 14 shows the results of a microPET scan of the two mice. FIG. 14 shows that [¹⁸F]compound 14 accumulated in the lungs of the LPS-treated mouse, but not in the control mouse. This difference in tracer accumulation is consistent with the difference in accumulation of iNOS described between LPS-treated and control mice in the preceeding examples.

All publications, patents, patent applications, and other references cited in this application are incorporated herein by reference in their entirety. Citation of a reference herein shall not be construed as an admission that such is prior art relevant to patentability of the present invention.

What is claimed is:

1. A radiolabeled 2-amino-4-alkyl-6-(haloalkyl)pyridine compound or salt thereof of structure 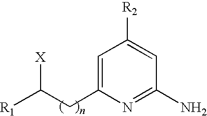
wherein R₁ is H or C₁₋₅ alkyl, X is a halogen, n is an integer from 0 to 5, and R₂ is C₁₋₅ alkyl, wherein the compound inhibits iNOS with an IC₅₀ of less than 140 nM when measured against SEITU, L-NIL and 2-AP as standards.

2. A radiolabeled 2-amino-4-alkyl-6-(haloalkyl)pyridine compound or salt thereof of structure 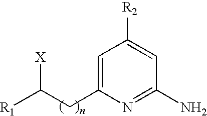
wherein R₁ is H or C₁₋₅ alkyl, X is a halogen, n is an integer from 0 to 5, and R₂ is C₁₋₅ alkyl, wherein the compound inhibits iNOS with an IC₅₀ less than that of any of SEITU, L-NIL or 2-AP and wherein at least one atom is a radioisotope.

3. A radiolabeled 2-amino-4-alkyl-6-haloalkylpyridine compound or salt thereof in accordance with claim 1, wherein the compound inhibits iNOS with an IC₅₀ of less than or equal to 57.6 nM, or about 57.6 nM, when measured against SEITU, L-NIL and 2-AP as standards.

4. A radiolabeled 2-amino-4-alkyl-6-haloalkylpyridine compound or salt thereof in accordance with claim 1, wherein the compound inhibits iNOS with an IC₅₀ of less than or equal to 54.3 nM, or about 54.3 nM, when measured against SEITU, L-NIL and 2-AP as standards.

5. A radiolabeled 2-amino-4-alkyl-6-haloalkylpyridine compound or salt thereof in accordance with claim 1, wherein R₁ is H or CH₃ and R₂ is CH₃.

6. A radiolabeled 2-amino-4-alkyl-6-haloalkylpyridine compound or salt thereof in accordance with claim 4, wherein R₁ is CH₃ and n=1.

7. A radiolabeled 2-amino-4-alkyl-6-haloalkylpyridine compound or salt thereof in accordance with claim 4, wherein R₁ is H and n=2.

8. A radiolabeled 2-amino-4-alkyl-6-haloalkylpyridine compound or salt thereof in accordance with claim 1, wherein R₁ is ¹¹CH₃.

9. A radiolabeled 2-amino-4-alkyl-6-haloalkylpyridine compound or salt thereof in accordance with claim 1, wherein R₂ is ¹¹CH₃.

10. A radiolabeled 2-amino-4-alkyl-6-haloalkylpyridine compound or salt thereof in accordance with claim 1, wherein the halogen is selected from an F, a Br and an I.

11. A radiolabeled 2-amino-4-alkyl-6-haloalkylpyridine compound or salt thereof in accordance with claim 10, wherein the F is an ¹⁸F.

12. A radiolabeled 2-amino-4-alkyl-6-haloalkylpyridine compound or salt thereof in accordance with claim 10, wherein the Br is a ⁷⁶Br.

13. A radiolabeled 2-amino-4-alkyl-6-haloalkylpyridine compound or salt thereof in accordance with claim 10, wherein the I is a ¹²³I.

14. A radiolabeled 2-amino-4-alkyl-6-haloalkylpyridine compound or salt thereof in accordance with claim 5, wherein n=1, R₁ is a CH₃, X is an ¹⁸F, and R₂ is a CH₃.

15. A radiolabeled 2-amino-4-alkyl-6-haloalkylpyridine compound or salt thereof in accordance with claim 5, wherein n=2, R₁ is an H, X is an ¹⁸F, and R₂ is CH₃.

16. A radiolabeled 2-amino-4-alkyl-6-haloalkylpyridine compound or salt thereof in accordance with claim 1, wherein the compound is selected from the group consisting of

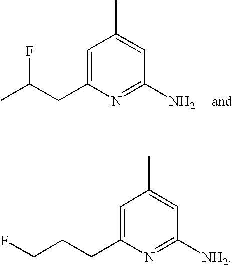

17. A radiolabeled 2-amino-4-alkyl-6-haloalkylpyridine compound or salt thereof in accordance with claim 1, wherein the compound is selected from the group consisting of

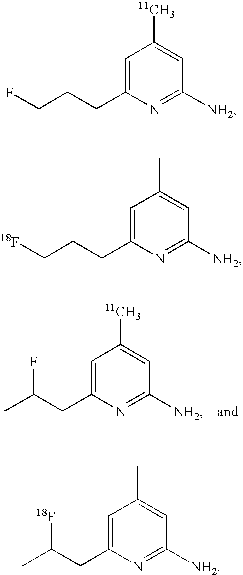

18. A salt comprising:
a radiolabeled 2-amino-4-alkyl-6-haloalkylpyridine compound in accordance with claim 1; and
a water soluble anion.

19. A salt in accordance with claim 18, wherein the water soluble anion is an anion of an organic acid.

20. A salt in accordance with claim 18, wherein the anion is an oxalate.

21. A method of imaging iNOS distribution in a mammal, the method comprising:
administering to the mammal a radiolabeled 2-amino-4-alkyl-6-(haloalkyl)pyridine compound or salt thereof, of claim 1; and
subjecting the mammal to PET scanning.

22. A method of imaging iNOS distribution in a mammal in accordance with claim 21, wherein the radiolabeled 2-amino-4-alkyl-6-(haloalkyl)pyridine compound is selected from the group consisting of

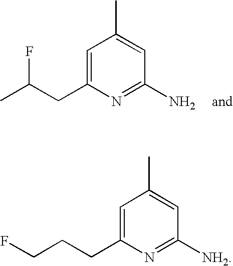

23. A method of imaging iNOS distribution in a mammal in accordance with claim 21, wherein the radiolabeled 2-amino-4-alkyl-6-(haloalkyl)pyridine compound is selected from the group consisting of [chemical structure]
